SEQUENCE LISTING (14 sequences)

SEQ ID NO: 1 - protein, 20 aa, Artificial Sequence
  Synthetic Construct
CCACATCTCCCTCCAGAAAA

SEQ ID NO: 2 - protein, 20 aa, Artificial Sequence
  Synthetic Construct
AGGGTCTGGGCCATAGAACT

SEQ ID NO: 3 - protein, 20 aa, Artificial Sequence
  Synthetic Construct
TCACAGCAGCACATCAACAA

SEQ ID NO: 4 - protein, 20 aa, Artificial Sequence
  Synthetic Construct
TGTCCTCATCCTGGAAGGTC

SEQ ID NO: 5 - protein, 20 aa, Artificial Sequence
  Synthetic Construct
TCCAGAAGGCCCTCAGACTA

SEQ ID NO: 6 - protein, 20 aa, Artificial Sequence
  Synthetic Construct
AGCATCTTCTCGACCCTGAA

SEQ ID NO: 7 - protein, 20 aa, Artificial Sequence
  Synthetic Construct
CCTCACAGCAACGAAGAACA

SEQ ID NO: 8 - protein, 20 aa, Artificial Sequence
  Synthetic Construct
ATCGAAAAGCCCGAAAGAGT

SEQ ID NO: 9 - protein, 20 aa, Artificial Sequence
  Synthetic Construct
CCAAGCCTTATCGGAAATGA

SEQ ID NO: 10 - protein, 20 aa, Artificial Sequence
  Synthetic Construct
TTTTCACAGGGGAGAAATCG

SEQ ID NO: 11 - protein, 20 aa, Artificial Sequence
  Synthetic Construct
CACTCTGGCCAACAACTTCA

SEQ ID NO: 12 - protein, 20 aa, Artificial Sequence
  Synthetic Construct
GCTTTCACCAAAGCGACTTC

SEQ ID NO: 13 - protein, 20 aa, Artificial Sequence
  Synthetic Construct
TTCACCACCATGGAGAAGGC

SEQ ID NO: 14 - protein, 20 aa, Artificial Sequence
  Synthetic Construct
GGCATGGACTGTGGTCATGA

(19) United States
(12) Patent Application Publication
(10) Pub. No.: US 2021/0100779 A1
(43) Pub. Date: Apr. 8, 2021

(54) COMPOSITIONS AND METHODS FOR TREATING RENAL INJURY

(71) Applicants: CASE WESTERN RESERVE UNIVERSITY, Cleveland, OH (US); INJE INDUSTRY ACADEMIC COOPERATION FOUNDATION, Gyeongsangnam-do (KP)

(72) Inventors: Sanford Markowitz, Cleveland, OH (US); Ki Beom Bae, Gyeongsangnam-do (KP); Hye Jung Kim, Gyeongsangnam-do (KP); Sun-Hee Kim, Gyeongsangnam-do (KP)

(21) Appl. No.: 17/044,888

(22) PCT Filed: Apr. 4, 2019
(86) PCT No.: PCT/US2019/025812

§ 371 Date: Oct. 2, 2020

Related U.S. Application Data
(60) Provisional application No. 62/652,769, filed on Apr. 4, 2018.

(51) Int. Cl. A61K 31/4365

(52) U.S. Cl. CPC A61K 31/4365

(57) ABSTRACT

A method for preventing or treating renal ischemia reperfusion injury or acute kidney injury associated with renal ischemia reperfusion injury in a subject in need thereof includes administering to the subject a therapeutically effective amount of a 15-PGDH inhibitor.

### RELATED APPLICATION

This application claims priority from U.S. Provisional Application No. 62/652,769, filed Apr. 4, 2018, the subject matter of which is incorporated herein by reference in its entirety.

### BACKGROUND

Acute kidney injury (AKI) is an important clinical problem associated with high rates of morbidity and mortality (1.7 million deaths annually). Considerable effort has been directed toward the development of preventive strategies for AM using various agents and animal models. Despite advances in prevention strategies, no specific treatment for AM has yet been developed.

The main causes of AM are hypoxia and oxidative stress due to renal ischemic reperfusion injury (IRI). During periods of transient reduction in renal blood flow (RBF), an insufficient oxygen supply can cause energy impairment (ATP depletion) in the renal outer medulla, resulting in the injury and death of the tubular epithelial cells due to acute tubular necrosis (ATN) and apoptosis. The inflammation due to oxygen-free radicals after reperfusion leads to the extension phase of ischemic AM. Resistance to hypoxia and the reduction of oxidative stress are treatment targets for ischemic AKI.

### SUMMARY

Embodiments described herein relate to compositions and methods of preventing, treating, or reducing the severity of renal ischemia reperfusion injury (IRI) or acute kidney injury (AKI). It was found that administration of a15-PGDH inhibitor to a subject prior to IRI can enhance renal PGE2 levels, induce renal vasodilation, and enhance resistance to hypoxia, resulting in a prophylactic and protective effect against ischemic AKI. Administration of a 15-PGDH inhibitor pre-IRI also improved renal hemodynamics, decreased induction of oxidative stress, reduced induction of inflammation, attenuated multiple markers of renal damage and preserved renal function. Accordingly, in some embodiments, compositions and methods of inhibiting 15-PDGH activity can be used to prevent, treat, or reduce the severity of IRI or AKI associated with IRI in a subject in need thereof.

In some embodiments, the 15-PGDH inhibitor can prevent or treat acute kidney injury associated with renal ischemia reperfusion injury.

In some embodiments, the amount of 15-PGDH inhibitor administered to the subject can be an amount effective to induce endogenous renal PGE2 levels of the subject.

In other embodiments, the amount of 15-PGDH inhibitor administered to the subject can be an amount effective to induce renal vasodilatation, enhance resistance to hypoxia, improve renal hemodynamics, decrease renal oxidative stress, reduce renal inflammation, and preserve renal function.

In other embodiments, the amount of 15-PGDH inhibitor administered to the subject is an amount effective to reduce malondialdehyde (MDA) and NGAL levels, attenuate medulla tubular damage, reduce medulla acute tubular necrosis (ATN) and apoptosis, reduces induction of high-mobility group box 1 (HMGB1) and pro-inflammatory cytokines, induce renal EP4 PGE2 receptors and A2A adenosine receptors in vascular smooth muscle cells that regulate renal arterioles, increase renal cAMP, AMP, and adenosine levels, and/or inhibit induction of creatinine and KIM-1.

In other embodiments, the 15-PGDH inhibitor can be administered to the subject before the ischemia reperfusion injury. For example, the 15-PGDH inhibitor is administered at a range of about 1 minute to about 72 hours before the ischemia reperfusion injury, about 10 minutes to about 48 hours before the ischemia reperfusion injury, or about 30 minutes to about 36 hours before the ischemia reperfusion injury.

In other embodiments, the 15-PGDH inhibitor can be administered at a time selected from the group consisting of 2 hours, 8 hours, 24 hours, and 26 hours before the ischemia reperfusion injury.

In some embodiments, the ischemia reperfusion injury is associated with an organ transplant, such as a kidney transplant, in the subject.

In other embodiments, the ischemia reperfusion injury is associated with cardiovascular surgery or sepsis.

In some embodiments, the 15-PGDH inhibitor can include a compound having the following formula (V):

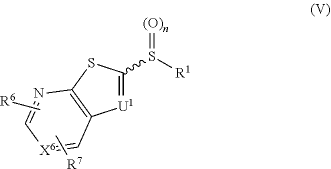

wherein n is 0-2

X⁶ is independently is N or CR^{c}

R¹, R⁶, R⁷, and R^{c} are each independently selected from the group consisting of hydrogen, substituted or unsubstituted C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms (wherein from 1-3 of the ring atoms is independently selected from N, NH, N(C₁-C₆ alkyl), NC(O)(C₁-C₆ alkyl), O, and S), C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺+N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂-O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂-NH2, —SO₂NY₂ (wherein Y is independently H, aryl or alkyl), phosphono (—P(O)(OH)₂), phosphonato (—P(O)(O⁻)₂), phosphinato (—P(O)(O⁻)), phospho (—PO²), phosphino (—PH₂), polyalkylethers, phosphates, phosphate esters, groups incorporating amino acids or other moieties expected to bear positive or negative charge at physiological pH, combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;

U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂-CH₂X, O—CH₂—CH₂X, CH₂—CH₂—CH₂X, O—CH₂—CH₂X, X, (wherein X═H, F, Cl, Br, or I), CN, (C═O)-R′, (C═O)N(R′)₂, O(CO)R′, COOR′ (wherein R′ is H or a lower alkyl group), and wherein R′ and R² may be linked to form a cyclic or polycyclic ring, wherein R³ and R⁴ are the same or different and are each selected from the group consisting of H, a lower alkyl group, O, (CH₂)ₙ₁R′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent; or a pharmaceutically acceptable salt, tautomer, or solvate thereof.

In some embodiments, the 15-PGDH inhibitor can inhibit the enzymatic activity of recombinant 15-PGDH at an IC₅₀ of less than 1 μM, or preferably at an IC₅₀ of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

### BRIEF DESCRIPTION OF THE DRAWINGS

FIGS. 1(A-K) illustrate plots showing 15-PGDH inhibition on renal IRI decreases the levels of renal injury biomarkers. (A) Arachidonic acid prostaglandin biosynthesis pathway and the biological activity of 15-PGDH inhibitor. (B) Endogenous PGE2 levels in the kidney tissue of 15-PGDH knockout (KO) and wild-type (WT) mice (n=5 per group). (C) Pharmacologic inhibition of 15-PGDH with SW033291 were confirmed by endogenous PGE2 levels in kidney tissue at 3 hours after i.p. injection of 2.5 or 5 mg/kg SW033291 or vehicle (n=5 per group). (D) PGE2 levels in kidney tissue at 1 and 3 hours after i.p. injection of 5 mg/kg SW033291 or vehicle (n=5 per group). (E-G) Renal damage according to the injury time. Data are means±SEM. *P<0.05 vs. corresponding WT or sham; **P<0.01 vs. corresponding WT or sham; ***P<0.001 vs. corresponding WT or sham. (H) Experimental setup. Mice were subjected to bilateral renal ischemia/reperfusion (I/R) and were injected with vehicle, SW033291, indomethacin, exogenous PGE1, or PGE2 at 1 hour before, immediately after, and 12 hours after renal IRI. Serum was collected after reperfusion for 24 hours. (I-K) Serum levels of NGAL, creatinine, and KIM-1. Renal function was evaluated at POD1 after renal IRI. Number of each group is 8-11. Data are means±SEM. *P<0.05 vs. corresponding IRI_vehicle; **P<0.01 vs. corresponding IRI_vehicle; ***P<0.001 vs. corresponding IRI_vehicle; #P<0.05 vs. corresponding sham; ##<0.01 vs. corresponding sham; ###P<0.001 vs. corresponding sham.

FIGS. 2(A-E) illustrate images and plots showing 15-PGDH inhibition ameliorates cell death and the inflammatory response in mice with ischemic AKI. Before and after renal IRI, mice were injected i.p. three times with vehicle, SW033291 (5 mg/kg) or indomethacin (5 mg/kg). Assessments were performed at POD1 after renal IRI. (A) Representative gross appearance of the left (Lt) and right (Rt) kidneys of mice injected with vehicle (IRI-vehicle), indomethacin (IRI-indomethacin), or SW033291 (IRI-SW033291) before and after renal IRI. Renal tissue congestion in the outer medulla is indicated by white arrows. (B) Representative image of tubular injury in the outer zone of the renal medulla (H&E staining, ×200 magnification). Scale bars, 500 μm; scale bar in the enlarged image, 50 μm. (C) Statistical analysis of tubular injury scores (n=20 per group). (D) Representative image of apoptosis in the outer zone of the renal medulla (TUNEL staining, ×400). Scale bars, 500 μm; scale bar in the enlarged image, 25 μm. (E) Statistical analysis of apoptosis (n=20 per group). *P<0.05 vs. corresponding IRI_vehicle; **P<0.01 vs. corresponding IRI_vehicle; ***P<0.001 vs. corresponding IRI_vehicle; #P<0.05 vs. corresponding sham; ##P<0.01 vs. corresponding sham.

FIGS. 3(A-H) illustrate a western blot and plots showing 15-PGDH inhibition ameliorates the inflammatory response in mice with ischemic AKI. (A) Western blots of HMGB1 (29 kDa) in kidney tissue (representative of three experiments). (B) Statistical analysis of HMGB1 levels in kidney tissue (n=9 per group). Pro-inflammatory cytokine mRNA by real time per (C-E) and protein levels by ELISA (F-G). Data are means±SEM. *P<0.05 vs. corresponding IRI_vehicle; **P<0.01 vs. corresponding IRI_vehicle; #P<0.05 vs. corresponding sham; ##<0.01 vs. corresponding sham.

FIGS. 4(A-D) illustrate images and plots showing 15-PGDH inhibition induces renal vasodilation in the outer medulla via the cAMP/AMP signaling pathway. To quantify vasodilation, the inner arteriole area in the outer medulla was identified by α-smooth muscle actin (α-SMA) staining. (A) Representative image of an arteriole in the outer zone of the renal medulla (×400 magnification). Zoomed images are enlargements of the outlined areas. (B) Statistical analysis of the inner arteriole area of the outer medulla. (C, D) Statistical analysis of cAMP and AMP levels in kidney tissue. Number of each group is 12˜18. Data are means±SEM. *P<0.05 vs. corresponding IRI_vehicle; **P<0.01 vs. corresponding IRI_vehicle; #P<0.05 vs. corresponding Sham; ##P<0.01 vs. corresponding sham. Scale bars, 500 μm; scale bar in the enlarged image, 50 μm.

FIGS. 5(A-G) illustrate plots and images showing 15-PGDH inhibitor promoted the expression of EP4 receptor in the renal arterioles in the outer medulla. (A-D) Statistical analysis of the EP receptors mRNA levels in kidney tissue by real-time PCR. Number of each group is 6˜10. (E) Western blots for EP4 (73 kDa) in kidney tissue (representative of three experiments). (F) Statistical analysis of EP4 levels in kidney tissue (n=6 per group). Data are means±SEM. *P<0.05 vs. corresponding IRI_vehicle; **P<0.01 vs. corresponding IRI_vehicle; #P<0.05 vs. corresponding Sham; ##P<0.01 vs. corresponding sham. (G) Representative confocal microscopy images for EP4 (green), α-SMA (red) and DAPI (blue) stained kidney sections. EP4 positive cells are seen in α-smooth muscle actin (α-SMA)-positive cells in the renal arteriole outer medulla (arrow). * indicates α-SMA positive renal arterioles in the outer medullar. Scale bars: 25 μm.

FIGS. 6(A-E) illustrate plots and images showing 15-PGDH inhibitor promoted adenosine production and upregulated the expression of A_{2A} receptor in the renal arterioles in the outer medulla. (A) Statistical analysis of adenosine levels in kidney tissue. (B) Statistical analysis of serum adenosine levels. Number of each group is 6˜10. (c) Western blots for A_{2A} (45 kDa) in kidney tissue (representative of three experiments). (D) Statistical analysis of A_{2A} levels in kidney tissue (n=9 per group). Data are means±SEM. *P<0.05 vs. corresponding IRI_vehicle; **P<0.01 vs. corresponding IRI_vehicle. (E) Representative confocal microscopy images for A_{2A} and α-smooth muscle actin (α-SMA) stained kidney sections. A_{2A} positive cells are seen in α-SMA positive cells in the renal arteriole outer medulla (arrow). * indicates α-SMA positive renal arterioles in the outer medullar. Scale bars: 25 μm.

FIGS. 7(A-D) illustrate schema and plots showing 15-PGDH Inhibitor Pretreatment Mitigates Renal Dysfunction after renal IRI. (A) Experimental setup for three different injection protocols. Mice were injected with vehicle or SW033291 (5 mg/kg) according to three different injection protocols. Renal function was assessed at POD1 after renal IRI. (B-D) Serum levels of NGAL, creatinine, and KIM-1. Number of each group is 9˜11. Data are means±SEM. *P<0.05 vs. corresponding IRI_vehicle; **P<0.01 vs. corresponding IRI_vehicle; ##P<0.01 vs. corresponding sham.

FIG. 8 illustrates a schematic showing the intrarenal vasodilatation mechanism by PGDH inhibitor in ischemic AKI. 15-PGDH inhibitor increases endogenous PGE2 by inhibiting degradation of PGE2 in ischemic AKI. Endogenous PGE2 induces vasodilation by activating EP4 receptors. Activation of EP4 increases intracellular cyclic AMP level in vascular smooth muscle cells. Increased cAMP is converted to adenosine substrate AMP, which in turn increases endovascular adenosine level. Adenosine activates A_{2A} to induce vasodilation. As a result, increased endogenous PGE2 by 15-PGDH inhibitor activates the EP4 receptor and increases adenosine, leading to vasodilatation of the intrarenal arteries. Alphabetical Abbreviation:15PGDH, 15-Hydroxyprostaglandin Dehydrogenase;A_{2A}, Adenosine A_{2A} receptor; AA, Arachidonic acids;ADO, Adenosine;AMP, Adenosine monophosphate;cAMP, Cyclic adenosine monophosphate;CD73, Ecto-5′-nucleotidase;COX2, Cyclooxigenase-2;EP4, Prostaglandin E2 receptor 4;ePDE, Extracellular phosphodiesterase;NSAIDs, Nonsteroidal anti-inflammatory drug;PGDH-i, 15-Hydroxyprostaglandin Dehydrogenase inhibitor;PGE2, Prostaglandin E2;RBC, Red blood cell.

FIGS. 9(A-B) illustrate an image and plot showing other vasodilators did not exert a renoprotective effect. Renal pathologic assessment was performed at POD1 after renal IRI. Mice were injected with vehicle (IRI-vehicle), SW033291 (IRI-SW033291), Eglandin (IRI-PGE1), or exogenous PGE2 (IRI-PGE2) before and after renal IRI. (A) Representative image of tubular injury in the outer zone of the renal medulla (H&E staining, ×200 magnification). (B) Statistical analysis of tubular injury scores. Number of each group is 20. Data are means±SEM. **P<0.01 vs. corresponding IRI-vehicle. Scale bars, 50 μm.

FIGS. 10(A-C) illustrate plots showing 15-PGDH inhibitor pretreatment exerted an anti-inflammatory effect in mice with ischemic AKI. Before or after renal IRI, mice were injected three times i.p. with vehicle, SW033291 (5 mg/kg) or indomethacin (5 mg/kg). (A-C) Real-time PCR was performed at POD1 after renal IRI. mRNA levels of IL-24, IL-10, and IL-4. Number of each group is 9. Data are means±SEM. *P<0.05 vs. corresponding IRI-vehicle.

FIGS. 11(A-H) illustrate plots showing 15-PGDH inhibitor pretreatment attenuates the increase of PGE2 level and renal damage after renal IRI. PGE2 levels in kidney tissue (A) and serum (B). (C, D) EP4 and A2A mRNA levels in kidney tissue. (E) MDA levels in kidney tissue. (F-H) NGAL, KIM-1, and creatinine levels in serum. Number of each group is 4˜8. Data are means±SEM. *P<0.05 vs. corresponding IRI_vehicle; **P<0.01 vs. corresponding IRI_vehicle; #P<0.05 vs. corresponding baseline; ##P<0.01 vs. corresponding baseline

FIGS. 12(A-C) illustrate plots showing 15-PGDH Inhibitor treatment is Non-Toxic and Promotes Recovery after Renal IRI. (A) Experimental setup for toxicity. Ischemic AKI induced by bilaterally clamping the renal arteries for 30 minutes, followed by reperfusion. The mice were injected with vehicle, SW033291 (5 mg/kg), or indomethacin (5 mg/kg) i.p. before and twice daily after renal IRI over 7 days. (n=10 per group). (B, C) Survival curves and body weight over 7 days. Kaplan-Meier analysis of survival stratified by AKI stage. Data are means±SEM. *P<0.05 vs. corresponding BI30_vehicle.

### DETAILED DESCRIPTION

While the following terms are believed to be well understood by one of ordinary skill in the art, the following definitions are set forth to facilitate explanation of the presently disclosed subject matter.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine,

N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the -OH radical.
“Imino” refers to the=NH substituent.
“Nitro” refers to the —NO₂ radical.
“Oxo” refers to the=0 substituent.
“Thioxo” refers to the=S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-Cs alkyl. A C₁-C₅ alkyl includes C5 alkyls, C4 alkyls, C3 alkyls, C2 alkyls and C1 alkyl (i.e., methyl). A C₁-C₆ alkyl includes all moieties described above for C₁-C5 alkyls but also includes C₆ alkyls. A C₁-C₁₀ alkyl includes all moieties described above for C₁-C₅ alkyls and C₁-C₆ alkyls, but also includes C7, C8, C9 and C10 alkyls. Similarly, a C₁-C₁₂ alkyl includes all the foregoing moieties, but also includes C11 and C12 alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C_{7,} C_{8,} C₉ and Cm alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C_{7,} C_{8,} C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and Rd is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}—R_{c} where R_{b} is an alkylene group as defined above and R_{c} is a heterocyclyl radical as defined above. Unless stated otherwise specifically in the specification, a heterocyclylalkyl group can be optionally substituted.

“Heterocyclylalkenyl” refers to a radical of the formula —R_{b}—R_{c} where R_{b} is an alkenylene group as defined above and R_{c} is a heterocyclyl radical as defined above. Unless stated otherwise specifically in the specification, a heterocyclylalkenyl group can be optionally substituted.

“Heterocyclylalkynyl” refers to a radical of the formula —R_{b}—R_{c} where R_{b} is an alkynylene group as defined above and R_{c} is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{B}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and R_{b} are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

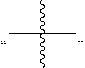

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

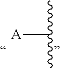

indicates that the chemical entity “A” is bonded to another chemical entity via the point of attachment bond. Furthermore, the specific point of attachment to the non-depicted chemical entity can be specified by inference. For example, the compound

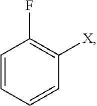

wherein X is

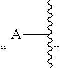

infers that the point of attachment bond is the bond by which X is depicted as being attached to the phenyl ring at the ortho position relative to fluorine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC_{50,}” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

All percentages and ratios used herein, unless otherwise indicated, are by weight.

The terms “gene expression” or “protein expression” includes any information pertaining to the amount of gene transcript or protein present in a sample, as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The terms “healthy” and “normal” are used interchangeably herein to refer to a subject or particular cell or tissue that is devoid (at least to the limit of detection) of a disease condition.

The term “nucleic acid” refers to polynucleotides such as deoxyribonucleic acid (DNA), and, where appropriate, ribonucleic acid (RNA). The term should also be understood to include analogues of either RNA or DNA made from nucleotide analogues, and, as applicable to the embodiment being described, single-stranded (such as sense or antisense) and double-stranded polynucleotides. In some embodiments, “nucleic acid” refers to inhibitory nucleic acids. Some categories of inhibitory nucleic acid compounds include antisense nucleic acids, RNAi constructs, and catalytic nucleic acid constructs. Such categories of nucleic acids are well-known in the art.

Embodiments described herein relate to compositions and methods of preventing, treating, or reducing the severity of renal ischemia reperfusion injury (IRI) or acute kidney injury (AM). It was found that administration of a15-PGDH inhibitor to a subject prior to ischemia reperfusion injury can enhance renal PGE2 levels, induce renal vasodilation, and enhance resistance to hypoxia, resulting in a prophylactic and protective effect against ischemic acute kidney injury. In particular, administration of a 15-PGDH inhibitor pre-IRI improved renal hemodynamics, decreased induction of oxidative stress, reduced induction of inflammation, attenuated multiple markers of renal damage and preserved renal function. Advantageously, the administration of a 15-PGDH inhibitor systemically to a subject to generate endogenous renal PGE2 showed greater effectiveness than systemic administration of PGE1 or PGE2.

Accordingly, in some embodiments compositions and methods of inhibiting 15-PDGH activity can be used to prevent, treat, or reduce the severity of ischemia reperfusion injury or acute kidney injury associated with ischemia reperfusion injury in a subject in need thereof.

In certain embodiments, the subject has been identified as having AKI based on the Acute Kidney Injury Network (AKIN) criteria or Risk/Injury/Failure/Loss/ESRD (RIFLE) criteria.

In another embodiment, the subject has been identified as having an elevated level of serum creatinine, plasma creatinine, urine creatinine, or blood urea nitrogen (BUN), compared to a healthy control subject.

In another embodiment, the subject has been identified as having an elevated level of serum or urine neutrophil gelatinase-associated lipocalin, serum or urine interleukin-18, serum or urine cystatin C, or urine MM-1, compared to a healthy control subject.

In some embodiments, the acute kidney injury is an ischemic acute kidney injury. In one embodiment, the subject is a human who has been identified as having reduced effective arterial volume. In one embodiment, the subject has been identified as having intravascular volume depletion (e.g., due to hemorrhage, gastrointestinal loss, renal loss, skin and mucous membrane loss, nephrotic syndrome, cirrhosis, or capillary leak). In one embodiment, the subject has been identified as having reduced cardiac output (e.g., due to cardiogenic shock, pericardial disease, congestive heart failure, valvular heart disease, pulmonary disease, or sepsis). In one embodiment, the subject has been identified as having systemic vasodilation (e.g., caused by cirrhosis, anaphylaxis, or sepsis). In one embodiment, the subject has been identified as having renal vasoconstriction (e.g., caused by early sepsis, hepatorenal syndrome, acute hypercalcemia, a drug, or a radiocontrast agent).

In some embodiments, the acute kidney injury is a nephrotoxic acute kidney injury. In one embodiment, the human subject has been exposed to a nephrotoxin. For example, the nephrotoxin can be a nephrotoxic drug selected from the group consisting of an antibiotic (e.g., an aminoglycoside), a chemotherapeutic agent (e.g., cis-platinum), a calcineurin inhibitor, amphotericin B, and a radiographic contrast agent. In another example, the nephrotoxin can be an illicit drug or a heavy metal.

In certain embodiments, the subject has undergone a trauma injury or a crush injury.

In certain embodiments, the subject will undergo or has undergone an organ transplant surgery (e.g., a kidney transplant surgery or heart transplant surgery).

In certain embodiments, the subject will undergo or has undergone a surgery complicated by hypoperfusion.

In certain embodiments, the subject will undergo or has undergone cardiothoracic surgery or a vascular surgery.

In certain embodiments, the subject will be taking or has taken medication (e.g., an anticholinergic) that interferes with normal emptying of the bladder.

In certain embodiments, the subject has benign prostatic hypertrophy or a cancer (e.g., prostate cancer, ovarian cancer, or colorectal cancer).

In certain embodiments, the subject has a kidney stone.

In certain embodiments, the subject has an obstructed urinary catheter.

In certain embodiments, the subject has taken a drug that causes or leads to crystalluria, a drug that causes or leads to myoglobinuria, or a drug that causes or leads to cystitis.

Other embodiments, described herein relate to a method for protecting a kidney from injury in a subject. The method involves administering to the subject an effective amount of 15-PGDH inhibitor to protect the subject's kidney from injury. In some embodiments, the subject has been or will be exposed to an ischemic or nephrotoxic insult. In some embodiments, the human subject has been exposed to oxidative damage (e.g., by free radicals such as reactive oxygen or nitrogen species.

Still further embodiments relate to a method for protecting a human subject's kidney from acute kidney injury during transplantation. The method involves administering to the subject an effective amount of 15-PGDH inhibitor to protect the subject's kidney from injury. In certain embodiments, the method further comprises administering to the human subject one or more doses of a 15-PGDH inhibitor before and/or after (e.g., 0.1, 0.2, 0.3, 0.4, 0.5, 1, 2, 3, 4, 5, 6, 7, 8, 9, 10, 11, 12, 24, 48, 72, 96,168 hours, or 1 week, 2 weeks, 3 weeks or 1 month) the organ transplantation.

In some embodiments, 15-PGDH inhibitors potentially used in preventing, treating, or reducing the severity of renal ischemia reperfusion injury (IRI) or acute kidney injury (AM) can be identified using assays in which putative inhibitor compounds are applied to cells expressing 15-PGDH and then the functional effects on 15-PGDH activity are determined. Samples or assays comprising 15-PGDH that are treated with a potential inhibitor are compared to control samples without the inhibitor to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%.

Agents tested as 15-PGDH inhibitors can be any small chemical molecule or compound. Typically, test compounds will be small chemical molecules, natural products, or peptides. The assays are designed to screen large chemical libraries by automating the assay steps and providing compounds from any convenient source to assays, which are typically run in parallel (e.g., in microtiter formats on microtiter plates in robotic assays).

In some embodiments, the 15-PGDH inhibitor can include a compound having the following formula (I):

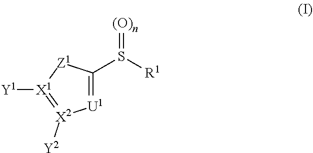

wherein n is 0-2;

Y¹, Y², and IV are the same or different and are each selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanate isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof, and

wherein Y¹ and Y² may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;

U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X,

O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent;

X¹ and X² are independently N or C, and wherein when X¹ and/or X² are N, Y¹ and/or Y², respectively, are absent;

Z¹ is O, S, CR^{a}R^{b} or NR^{a}, wherein R^{a} and R^{b} are independently H or a C₁₋₈ alkyl, which is linear, branched, or cyclic, and which is unsubstituted or substituted;

or a pharmaceutically acceptable salt, tautomer, or solvate thereof.

In other embodiments, the 15-PGDH inhibitor can include a compound having the following formula (II):

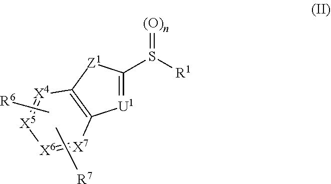

wherein n is 0-2

X⁴, X⁵, X⁶, and X⁷ are independently N or CR^{c};

R¹, R⁶, R⁷, and W are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanate (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂-OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;

U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, Cl, Br, or I), CN, (C═O)—R′, (C═O)N(R′)₂, COOR′ (wherein R′ is H or a lower alkyl group), and R³ or R⁴ may be absent;

Z¹ is O, S, CR^{a}R^{b} or NR^{a}, wherein R^{a} and R^{b} are independently H or a C₁₋₈ alkyl, which is linear, branched, or cyclic, and which is unsubstituted or substituted;

or a pharmaceutically acceptable salt, tautomer, or solvate thereof.

In yet other embodiments, the 15-PGDH inhibitor can include a compound having the following formula (III) or (IV):

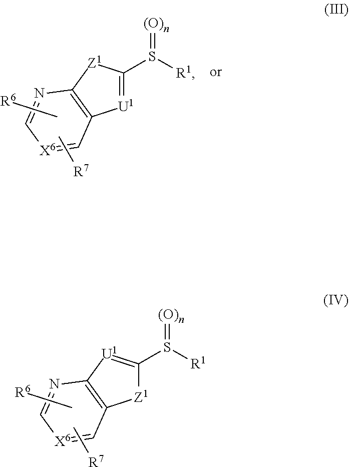

wherein n is 0-2

X⁶ is independently is N or CR^{c};

R¹, R⁶, R⁷, and R^{c} are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanate isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)—alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂-NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [OP(O)(OR)₂ where R═H, methyl or other alkyl], groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;

U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent;

Z¹ is O, S, CR^{a}R^{b} or NR^{a}, wherein R^{a} and R^{b} are independently H or a C₁₋₈ alkyl, which is linear, branched, or cyclic, and which is unsubstituted or substituted;

or a pharmaceutically acceptable salt, tautomer, or solvate thereof.

In some embodiments, R¹ is selected from the group consisting of branched,

linear, or cyclic alkyl,

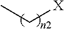

wherein n₂=0-6 and X is any of the following: CF_{y}H_{z} (y+z=3), CCl_{y}H_{z} (y+z=3), OH, OAc, OMe, R⁷¹, OR⁷², CN, N(R⁷³)₂,

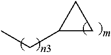

(n₃=0-5, m=1-5), and

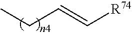

(n₄=0-5).

In other embodiments, R⁶ and R⁷ can each independently be one of the following:

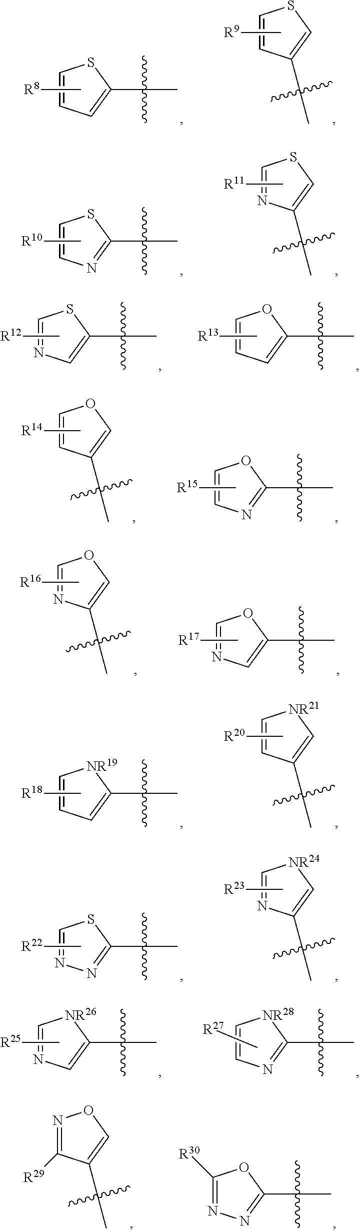

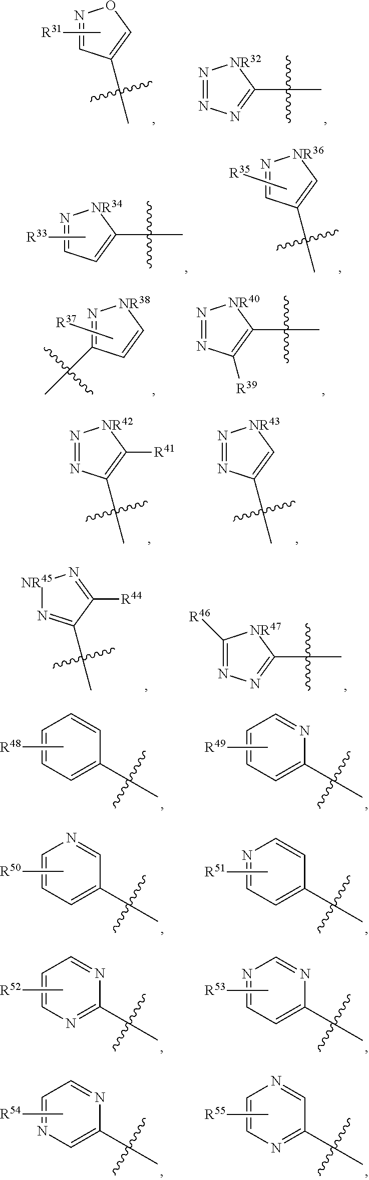

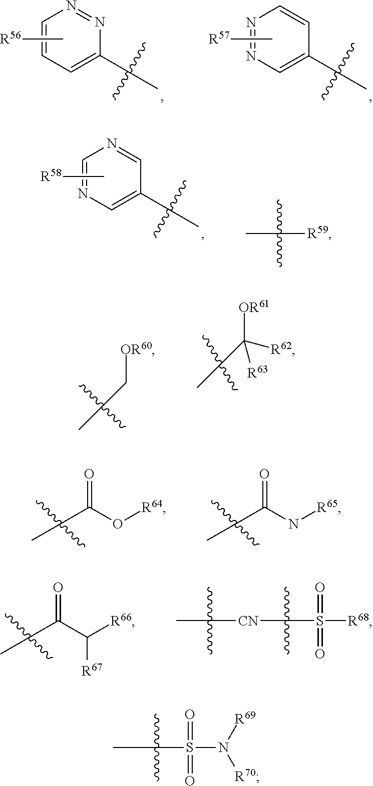

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, and R⁷⁴ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamide (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂-NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof, or a pharmaceutically acceptable salt, tautomer, or solvate thereof.

In still other embodiments, R⁶ and R⁷ can independently be a group that improves aqueous solubility, for example, a phosphate ester (—OPO₃H₂), a phenyl ring linked to a phosphate ester (—OPO₃H₂), a phenyl ring substituted with one or more methoxyethoxy groups, or a morpholine, or an aryl or heteroaryl ring substituted with such a group.

In other embodiments, the 15-PGDH inhibitor can include a compound having the following formula (V):

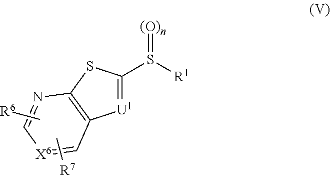

wherein n is 0-2

X⁶ is independently is N or CR^{c}

R¹, R⁶, R⁷, and W are each independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanate (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (-CR=N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (-S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R=H, methyl or other alkyl], groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;

U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent;

and pharmaceutically acceptable salts thereof.

In some embodiments, R¹ is selected from the group consisting of branched, linear, or cyclic alkyl,

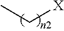

wherein n₂=0-6 and X is any of the following: CF_{y}H_{z} (y+z=3), CCl_{y}H_{z} (y+z=3), OH, OAc, OMe, R⁷¹, OR⁷², CN, N(R⁷³)₂,

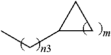

(n₃=0-5, m=1-5), and

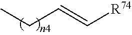

(n₄=0-5).

In other embodiments, R⁶ and R⁷ can each independently be one of the following:

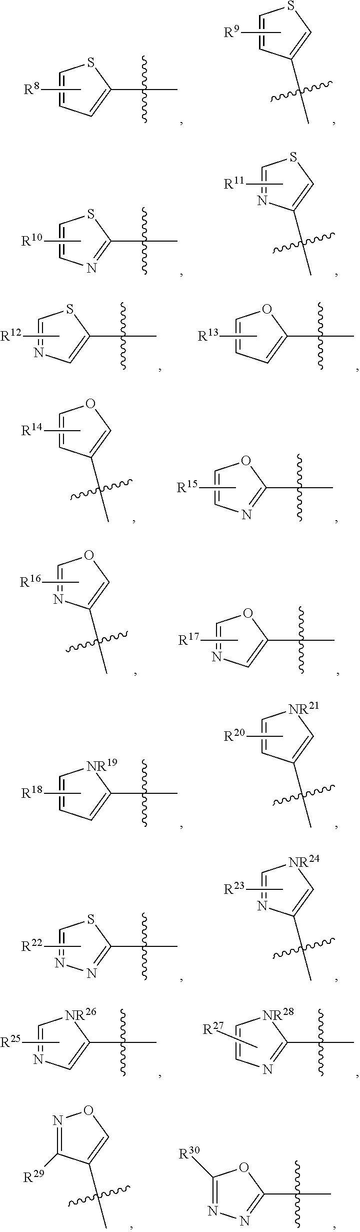

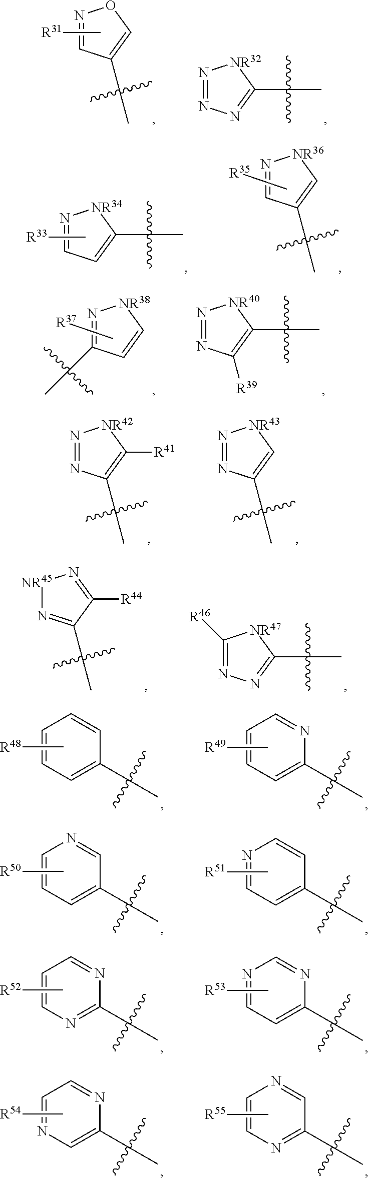

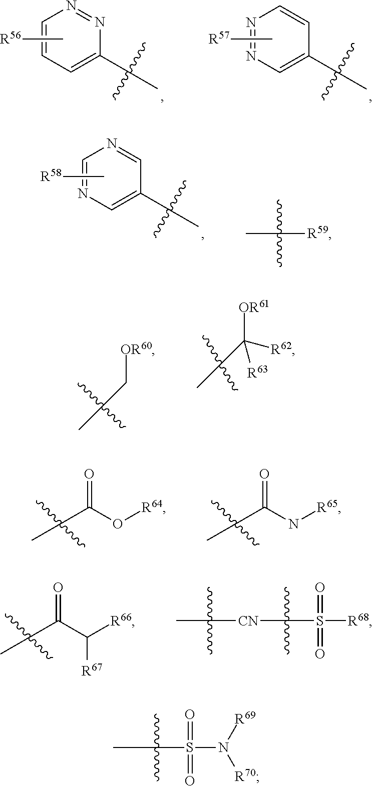

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, and R⁷⁴, are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(0)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)-NH-aryl), thiocarbamoyl (—(CS)—NH₂), carbamide (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂-OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof, or a pharmaceutically acceptable salt, tautomer, or solvate thereof.

In still other embodiments, R⁶ and R⁷ can independently be a group that improves aqueous solubility, for example, a phosphate ester (—OPO₃H₂), a phenyl ring linked to a phosphate ester (—OPO₃H₂), a phenyl ring substituted with one or more methoxyethoxy groups, or a morpholine, or an aryl or heteroaryl ring substituted with such a group.

Examples of compounds having formulas (I), (II), (III), (IV) and (V), are selected from are described in U.S. Patent Application Publication Nos. 2015/0072998, 2017/0165241, 2017/0173028, 2018/0118756, and WO2018/218251, all of which are incorporated by reference in their entirety.

In certain embodiments, the 15-PGDH inhibitor having formula (I), (II), (III), (IV), and (V) can be selected that can ia) at 2.5 μM concentration, stimulate a Vaco503 reporter cell line expressing a 15-PGDH luciferase fusion construct to a luciferase output level of greater than 70 (using a scale on which a value of 100 indicates a doubling of reporter output over baseline); iia) at 2.5 μM concentration stimulate a V9m reporter cell line expressing a 15-PGDH luciferase fusion construct to a luciferase output level of greater than 75; iiia) at 7.5 μM concentration stimulate a LS174T reporter cell line expressing a 15-PGDH luciferase fusion construct to a luciferase output level of greater than 70; and iva) at 7.5 μM concentration, does not activate a negative control V9m cell line expressing TK-renilla luciferase reporter to a level greater than 20; and va) inhibits the enzymatic activity of recombinant 15-PGDH protein at an IC₅₀ of less than 1 μM.

In other embodiments, the 15-PGDH inhibitor can ib) at 2.5 μM concentration, stimulate a Vaco503 reporter cell line expressing a 15-PGDH luciferase fusion construct to increase luciferase output; iib) at 2.5 μM concentration stimulate a V9m reporter cell line expressing a 15-PGDH luciferase fusion construct to increase luciferase output; iiib) at 7.5 μM concentration stimulate a LS174T reporter cell line expressing a 15-PGDH luciferase fusion construct to increase luciferase output; ivb) at 7.5 μM concentration, does not activate a negative control V9m cell line expressing TK-renilla luciferase reporter to a luciferase level greater than 20% above background; and vb) inhibits the enzymatic activity of recombinant 15-PGDH protein at an IC₅₀ of less than 1 μM.

In other embodiments, the 15-PGDH inhibitor can inhibit the enzymatic activity of recombinant 15-PGDH at an IC₅₀ of less than 1 μM, or preferably at an IC₅₀ of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

In some embodiments, the 15-PGDH inhibitor that can be administered to tissue or blood of a subject at an amount effective to inhibit the activity of a short chain dehydrogenase enzyme. The 15-PGDH inhibitor that can be administered to tissue or blood of a subject at an amount effective to increase prostaglandin levels in the tissue or blood.

In some embodiments, a 15-PGDH inhibitor having formula (V) can include a compound with the following formula (VI):

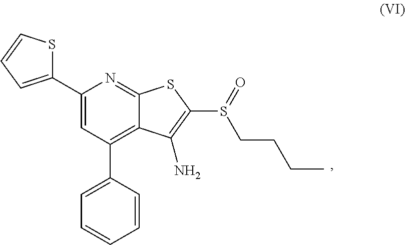 [SMILES: CCCCS(=O)c1sc2nc(-c3cccs3)cc(-c3ccccc3)c2c1N]

or a pharmaceutically acceptable salt, tautomer, or solvate thereof.

Advantageously, the 15-PDGH inhibitor having formula (VI) (SW033291) was found to: i) inhibit recombinant 15-PGDH at 1 nM concentration; ii) inhibit 15-PGDH in cell lines at 100 nM concentration, iii) increase PGE₂ production by cell lines; iv) is chemically stable in aqueous solutions over broad pH range; v) is chemically stable when incubated with hepatocyte extracts, vi) is chemically stable when incubated with hepatocyte cell lines; vii) shows 253 minutes plasma half-life when injected IP into mice; and viii) shows no immediate toxicity over 24 hours when injected IP into mice at 0.6 μmole/per mouse and at 1.2 μmole/per mouse and also no toxicity when injected IP into mice at 0.3 μmole/per mouse twice daily for 21 days.

In other embodiments, a 15-PGDH inhibitor having formula (VI) can include a compound with the following formula (VIa):

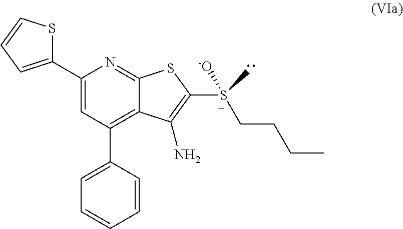

or a pharmaceutically acceptable salt, tautomer, or solvate thereof.

In still other embodiments, a 15-PGDH inhibitor having formula (VI) can include a compound with the following formula (VIb):

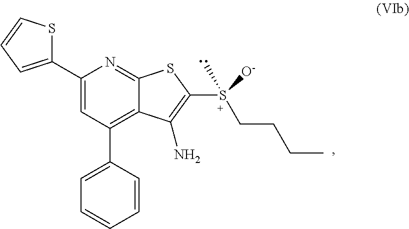

or a pharmaceutically acceptable salt, tautomer, or solvate thereof.

In other embodiments, the 15-PDHG inhibitor can comprise a (+) or (−) optical isomer of a 15-PGDH inhibitor having formula (VI). In still other embodiments, the 15-PDHG inhibitor can comprise a mixture at least one of a (+) or (−) optical isomer of a 15-PGDH inhibitor having formula (VI). For example, the 15-PGDH inhibitor can comprise a mixture of: less than about 50% by weight of the (−) optical isomer of a 15-PGDH inhibitor having formula (VI) and greater than about 50% by weight of the (+) optical isomer of a 15-PGDH inhibitor having formula (VI), less than about 25% by weight of the (−) optical isomer of a 15-PGDH inhibitor having formula (VI) and greater than about 75% by weight of the (+) optical isomer of a 15-PGDH inhibitor having formula (VI), less than about 10% by weight of the (−) optical isomer of a 15-PGDH inhibitor having formula (VI) and greater than about 90% by weight of the (+) optical isomer of a 15-PGDH inhibitor having formula (VI), less than about 1% by weight of the (−) optical isomer of a 15-PGDH inhibitor having formula (VI) and greater than about 99% by weight of the (+) optical isomer of a 15-PGDH inhibitor having formula (VI), greater than about 50% by weight of the (−) optical isomer of a 15-PGDH inhibitor having formula (VI) and less than about 50% by weight of the (+) optical isomer of a 15-PGDH inhibitor having formula (VI), greater than about 75% by weight of the (−) optical isomer of a 15-PGDH inhibitor having formula (VI) and less than about 25% by weight of the (+) optical isomer of a 15-PGDH inhibitor having formula (VI), greater than about 90% by weight of the (−) optical isomer of a 15-PGDH inhibitor having formula (VI) and less than about 10% by weight of the (+) optical isomer of a 15-PGDH inhibitor having formula (VI), or greater than about 99% by weight of the (−) optical isomer of a 15-PGDH inhibitor having formula (VI) and less than about 1% by weight of the (+) optical isomer of a 15-PGDH inhibitor having formula (VI).

In a still further embodiment, the 15-PDGH inhibitor can consist essentially of or consist of the (+) optical isomer of a 15-PGDH inhibitor having formula (VI). In yet another embodiment, the PDGH inhibitor can consist essentially of or consist of the (−) optical isomer of a 15-PGDH inhibitor having formula (VI).

In other embodiments, a 15-PGDH inhibitor having formula (V) can include a compound with the following formula (VII):

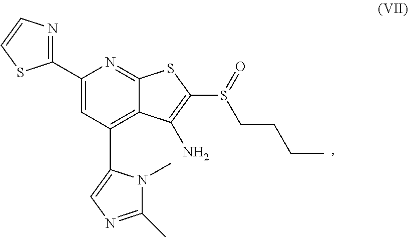 [SMILES: CCCCS(=O)c1sc2nc(-c3nccs3)cc(-c3cnc(C)n3C)c2c1N]

and pharmaceutically acceptable salts thereof.

Advantageously, the 15-PDGH inhibitor having formula (VII) was found to: i) inhibit recombinant 15-PGDH at 3 nM concentration; ii) increase PGE₂ production by cell lines at 20 nM; iii) is chemically stable in aqueous solutions over broad pH range; iv) is chemically stable when incubated with mouse, rat and human liver extracts, v) shows 33 minutes plasma half-life when injected IP into mice; viii) shows no immediate toxicity over 24 hours when injected IP into mice at 50 mg/kg body weight, and ix) is soluble in water (pH=3) at 1 mg/mL.

In other embodiments, a 15-PGDH inhibitor having formula (VII) can include a compound with the following formula (VIIa):

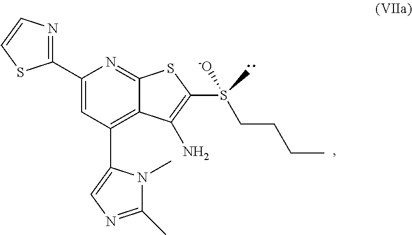

and pharmaceutically acceptable salts thereof.

In still other embodiments, a 15-PGDH inhibitor having formula (VII) can include a compound with the following formula (VIIb):

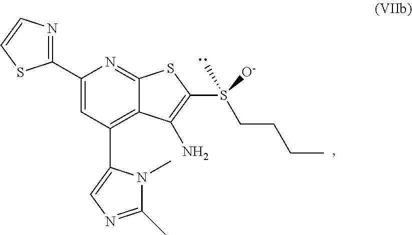

and pharmaceutically acceptable salts thereof.

In other embodiments, the 15-PDHG inhibitor can comprise a (+) or (−) optical isomer of a 15-PGDH inhibitor having formula (VII). In still other embodiments, the 15-PDHG inhibitor can comprise a mixture at least one of a (+) or (−) optical isomer of a 15-PGDH inhibitor having formula (VII). For example, the 15-PGDH inhibitor can comprise a mixture of: less than about 50% by weight of the (−) optical isomer of a 15-PGDH inhibitor having formula (VII) and greater than about 50% by weight of the (+) optical isomer of a 15-PGDH inhibitor having formula (VII), less than about 25% by weight of the (−) optical isomer of a 15-PGDH inhibitor having formula (VII) and greater than about 75% by weight of the (+) optical isomer of a 15-PGDH inhibitor having formula (VII), less than about 10% by weight of the (−) optical isomer of a 15-PGDH inhibitor having formula (VII) and greater than about 90% by weight of the (+) optical isomer of a 15-PGDH inhibitor having formula (VII), less than about 1% by weight of the (−) optical isomer of a 15-PGDH inhibitor having formula (VII) and greater than about 99% by weight of the (+) optical isomer of a 15-PGDH inhibitor having formula (VII), greater than about 50% by weight of the (−) optical isomer of a 15-PGDH inhibitor having formula (VII) and less than about 50% by weight of the (+) optical isomer of a 15-PGDH inhibitor having formula (VII), greater than about 75% by weight of the (−) optical isomer of a 15-PGDH inhibitor having formula (VII) and less than about 25% by weight of the (+) optical isomer of a 15-PGDH inhibitor having formula (VII), greater than about 90% by weight of the (−) optical isomer of a 15-PGDH inhibitor having formula (VII) and less than about 10% by weight of the (+) optical isomer of a 15-PGDH inhibitor having formula (VII), or greater than about 99% by weight of the (−) optical isomer of a 15-PGDH inhibitor having formula (VII) and less than about 1% by weight of the (+) optical isomer of a 15-PGDH inhibitor having formula (VII).

In a still further embodiment, the 15-PDGH inhibitor can consist essentially of or consist of the (+) optical isomer of a 15-PGDH inhibitor having formula (VII). In yet another embodiment, the PDGH inhibitor can consist essentially of or consist of the (−) optical isomer of a 15-PGDH inhibitor having formula (VII).

It will be appreciated that the other 15-PGDH inhibitors can be used in the methods described herein. These other 15-PGDH inhibitors can include known 15-PGDH inhibitors including, for example, tetrazole compounds of formulas (I) and (II), 2-alkylideneaminooxyacetamidecompounds of formula (I), heterocyclic compounds of formulas (VI) and (VII), and pyrazole compounds of formula (III) described in U.S. Patent Application Publication No. 2006/0034786 and U.S. Pat. No. 7,705,041; benzylidene-1,3-thiazolidine compounds of formula (I) described in U.S. Patent Application Publication No. 2007/0071699; phenylfurylmethylthiazolidine-2,4-dione and phenylthienylmethylthiazolidine-2,4-dione compounds described in U.S. Patent Application Publication No. 2007/0078175; thiazolidenedione derivatives described in U.S. Patent Application Publication No. 2011/0269954; phenylfuran, phenylthiophene, or phenylpyrrazole compounds described in U.S. Pat. No. 7,294,641, 5-(3,5-disubstituted phenylazo)-2-hydroxybenzene-acetic acids and salts and lactones described in U.S. Pat. No. 4,725,676, and azo compounds described in U.S. Pat. No. 4,889,846.

Still other examples are described in the following publications: Seo S Y et al. Effect of 15-hydroxyprostaglandin dehydrogenase inhibitor on wound healing. Prostaglandins Leukot Essent Fatty Acids. 2015;97:35-41. doi: 10.1016/j.plefa.2015.03.005. PubMed PMID: 25899574; Piao YL et al. Wound healing effects of new 15-hydroxyprostaglandin dehydrogenase inhibitors. Prostaglandins Leukot Essent Fatty Acids. 2014;91(6):325-32. doi: 10.1016/j.plefa.2014.09.011. PubMed PMID: 25458900; Choi D et al. Control of the intracellular levels of prostaglandin E(2) through inhibition of the 15-hydroxyprostaglandin dehydrogenase for wound healing. Bioorg Med Chem. 2013;21(15):4477-84. doi: 10.1016/j.bmc.2013.05.049. PubMed PMID: 23791868; Wu Y et al. Synthesis and biological evaluation of novel thiazolidinedione analogues as 15-hydroxyprostaglandin dehydrogenase inhibitors. J Med Chem. 2011;54(14):5260-4. Epub 2011/06/10. doi: 10.1021/jm200390u. PubMed PMID: 21650226; Duveau DY et al. Structure-activity relationship studies and biological characterization of human NAD(+)-dependent 15-hydroxyprostaglandin dehydrogenase inhibitors. Bioorg Med Chem Lett. 2014;24(2):630-5. doi: 10.1016/j.bmc1.2013.11.081. PubMed PMID: 24360556; PMCID: PMC_{3970110;} Duveau DY et al. Discovery of two small molecule inhibitors, ML387 and ML388, of human NAD+-dependent 15-hydroxyprostaglandin dehydrogenase. Probe Reports from the NIH Molecular Libraries Program. Bethesda (MD)2010; Wu Y et al. Synthesis and SAR of thiazolidinedione derivatives as 15-PGDH inhibitors. Bioorg Med Chem. 2010;18(4):1428-33. doi: 10.1016/j.bmc.2010.01.016. PubMed PMID: 20122835; Wu Y et al. Synthesis and biological evaluation of novel thiazolidinedione analogues as 15-hydroxyprostaglandin dehydrogenase inhibitors. J Med Chem. 2011;54(14):5260-4. Epub 2011/06/10. doi: 10.1021/jm200390u. PubMed PMID: 21650226; Jadhav A et al. Potent and selective inhibitors of NAD+-dependent 15-hydroxyprostaglandin dehydrogenase (HPGD). Probe Reports from the NIH Molecular Libraries Program. Bethesda (Md.)2010; Niesen FH et al. High-affinity inhibitors of human NAD-dependent 15-hydroxyprostaglandin dehydrogenase: mechanisms of inhibition and structure-activity relationships. PLoS One. 2010;5(11):e13719. Epub 2010/11/13. doi: 10.1371/journal.pone.0013719. PubMed PMID: 21072165; PMCID: 2970562; Michelet, J. et al. Composition comprising at least one 15-PGDH inhibitor. US20080206320 Al, 2008; and Rozot, R et al. Care/makeup compositions comprising a 2-alkylideneaminooxyacetamide compound for stimulating the growth of the hair or eyelashes and/or slowing loss thereof. U.S. Pat. No. 7,396,525 B2, 2008.

The 15-PGDH inhibitors described herein can be used to treat, prevent, or reduce the symptoms or severity of acute kidney injury in a subject (e.g. a human subject) in need thereof. The 15-PGDH inhibitors are also useful in preventing the development of chronic kidney disease in a subject in need thereof. In certain embodiments, the 15-PGDH inhibitors are useful in preventing the development of chronic kidney disease in a subject in need thereof following an insult that can cause or causes acute kidney injury. In addition, the 15-PGDH inhibitors described herein can be used in methods for protecting a kidney from acute or chronic kidney injury in a subject in need thereof. Furthermore, the 15-PGDH inhibitors described herein can be used in methods for treating patients with renal insufficiency or renal failure, attributable at least in part to use of a drug or chemical.

Acute kidney injury is commonly divided into two major categories based on the type of insult. The first category is ischemic acute kidney injury (alternatively referred to as kidney hypoperfusion) and the second category is nephrotoxic acute kidney injury. The former results from impaired blood flow (kidney hypoperfusion) and oxygen delivery to the kidney; whereas, the latter results from a toxic insult to the kidney. Both of these categories of insults can lead to a secondary condition called acute tubular necrosis (ATN).

The most common causes of ischemic acute kidney injury are intravascular volume depletion, reduced cardiac output, systemic vasodilatation, and renal vasoconstriction. Intravascular volume depletion can be caused by hemorrhage (e.g., following surgery, postpartum, or trauma); gastrointestinal loss (e.g., from diarrhea, vomiting, nasogastric loss); renal losses (e.g., caused by diuretics, osmotic diuresis, diabetes insipidus); skin and mucous membrane losses (e.g., burns, hyperthermia); nephrotic syndrome; cirrhosis; or capillary leak. Reduced cardiac output can be due to cardiogenic shock, pericardial disease (e.g. restrictive, constrictive, tamponade), congestive heart failure, valvular heart disease, pulmonary disease (e.g., pulmonary hypertension, pulmonary embolism), or sepsis. Systemic vasodilation can be the result of cirrhosis, anaphylaxis, or sepsis. Finally, renal vasoconstriction can be caused by early sepsis, hepatorenal syndrome, acute hypercalcemia, drug-related (e.g., norepinephrine, vasopressin, nonsteroidal anti-inflammatory drugs, angiotensin-converting enzyme inhibitors, calcineurin inhibitors), or use of a radiocontrast agent. The 15-PGDH inhibitors described herein can be used to treat or reduce the symptoms or severity of acute kidney injury or any other kidney injury caused by any of the above mentioned causes of ischemic acute kidney injury. In addition, the 15-PGDH inhibitors thereof described herein can be used to prevent the development of acute kidney injury or any other kidney injury following exposure to the above-mentioned causes of ischemic acute kidney injury.

Nephrotoxic acute kidney injury is often associated with exposure to a nephrotoxin such as a nephrotoxic drug. Examples of nephrotoxic drugs include an antibiotic (e g , aminoglycosides such as gentamicin), a chemotherapeutic agent (e.g., cis-platinum), a calcineurin inhibitor (e.g., tacrolimus, cyclosporine), cephalosporins such as cephaloridine, cyclosporin, pesticides (e.g., paraquat), environmental contaminants (e.g., trichloroethylene, dichloroacetylene), amphotericin B, puromcyin, aminonucleoside (PAN), a radiographic contrast agent (e.g., acetrizoate, diatrizoate, iodamide, ioglicate, iothalamate, ioxithalamate, metrizoate, metrizamide, iohexol, iopamidol, iopentol, iopromide, and ioversol), a nonsteroidal anti-inflammatory, an anti-retroviral, an immunosuppressant, an oncological drug, or an ACE inhibitor. A nephrotoxin can be, for example, a trauma injury, a crush injury, an illicit drug, analgesic abuse, a gunshot wound, or a heavy metal. The 15-PGDH inhibitors described herein can be used to treat or reduce the symptoms or severity of acute kidney injury or any other kidney injury caused by any of the above mentioned causes of nephrotoxic acute kidney injury. In addition, the 15-PGDH inhibitors described herein can be used to prevent the development of acute kidney injury or any other kidney injury following exposure to the above mentioned causes of nephrotoxic acute kidney injury.

In certain embodiments, the 15-PGDH inhibitors described herein can be used to prevent the development of ATN following exposure to an insult such as ischemia or nephrotoxins/nephrotoxic drugs. In certain embodiments, the 15-PGDH inhibitors described herein can be used to treat or reduce the symptoms or severity of ATN following ischemia or exposure to nephrotoxins/nephrotoxic drugs.

In certain embodiments, the 15-PGDH inhibitors described herein can be used to prevent a drop in glomerular filtration following ischemia or exposure to nephrotoxins/nephrotoxic drugs. In some embodiments, the 15-PGDH inhibitors can be used to prevent tubular epithelial injury and/or necrosis following ischemia or exposure to nephrotoxins/nephrotoxic drugs. In some embodiments, the 15-PGDH inhibitors can be used to decrease the microvascular permeability, improve vascular tone, and/or reduce inflammation of endothelial cells. In other embodiments, the 15-PGDH inhibitors described herein can be used to restore blood flow in the kidney following ischemia or exposure to nephrotoxins/nephrotoxic drugs. In further embodiments, the 15-PGDH inhibitors described herein can be used to prevent chronic renal failure.

The 15-PGDH inhibitors described herein can also be used to treat or prevent acute kidney injury resulting from surgery complicated by hypoperfusion. In certain specific embodiments, the surgery is one of cardiac surgery, major vascular surgery, major trauma, or surgery associated with treating a gunshot wound. In one embodiment, the cardiac surgery is coronary artery bypass grafting (CABG). In another embodiment, the cardiac surgery is valve surgery.

In some embodiments, the 15-PGDH inhibitors described herein can be used to treat or prevent acute kidney injury following organ transplantation such as kidney transplantation or heart transplantation.

In some embodiments, the 15-PGDH inhibitors described herein can be used to treat or prevent acute kidney injury following reduced effective arterial volume and kidney hypoperfusion.

In some embodiments, the 15-PGDH inhibitors described herein can be used to treat or prevent acute kidney injury in a subject who is taking medication (e.g., an anticholinergic) that interferes with normal emptying of the bladder. In certain embodiments, the 15-PGDH inhibitors described herein can be used to treat or prevent acute kidney injury in a subject who has an obstructed urinary catheter. In some embodiments, the 15-PGDH inhibitors described herein can be used to treat or prevent acute kidney injury in a subject who is taking a drug that causes crystalluria. In some embodiments, the 15-PGDH inhibitors described herein can be used to treat or prevent acute kidney injury in a subject who is taking a drug that causes or leads to myoglobinuria. In some embodiments, the 15-PGDH inhibitors described herein can be used to treat or prevent acute kidney injury in a subject who is taking a drug that causes or leads to cystitis.

In some embodiments, the 15-PGDH inhibitors described herein can be used to treat or prevent acute kidney injury in a subject who has benign prostatic hypertrophy or prostate cancer.

In some embodiments, the 15-PGDH inhibitors described herein can be used to treat or prevent acute kidney injury in a subject who has a kidney stone.

In some embodiments, the 15-PGDH inhibitors described herein can be used to treat or prevent acute kidney injury in a subject who has an abdominal malignancy (e.g., ovarian cancer, colorectal cancer).

In certain embodiments, the 15-PGDH inhibitors described herein can be used to treat or prevent acute kidney injury, wherein sepsis does not cause or result in the acute kidney injury.

Acute kidney injury typically occurs within hours to days following the original insult (e.g., ischemia or nephrotoxin insult). Thus, 15-PGDH inhibitors described herein can be administered before the insult, or within an hour to 30 days (e.g., 0.5 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 15 days, 20 days, 25 days, 28 days, or 30 days) after the insult (e.g., a surgery or nephrotoxin insult described herein).

A subject can be determined to have, or have the risk of developing, acute kidney injury based on, e.g., the Risk Injury Failure Loss ESRD (RIFLE) criteria or the Acute Kidney Injury Network criteria (Bagshaw et al., Nephrol. Dial. Transplant., 23 (5):1569-1574 (2008); Lopes et al., Clin. Kidney J., 6(1):8-14 (2013)).

In certain embodiments, the methods of this disclosure involve determining measuring the levels of one or more of: serum, plasma or urine creatinine or blood urea nitrogen (BUN); measuring the levels of serum or urine neutrophil gelatinase-associated lipocalin (NGAL), serum or urine interleukin-18 (IL-18), serum or urine cystatin C, or urine KIM-1, compared to a healthy control subject, to assess whether the subject has, or has a risk of developing, acute kidney injury.

The efficacy of the 15-PGDH inhibitors can be assessed in various animal models. Animal models for acute kidney injury include those disclosed in e.g., Heyman et al., Contrin. Nephrol., 169:286-296 (2011); Heyman et al., Exp. Opin. Drug Disc., 4(6): 629-641 (2009); Morishita et al., Ren. Fail., 33(10):1013-1018 (2011); Wei Q et al., Am. J. Physiol. Renal Physiol., 303(11):F1487-94 (2012).

The efficacy of treatments may be measured by a number of available diagnostic tools, including physical examination, blood tests, measurements of blood systemic and capillary pressure, proteinuria (e.g., albuminuria), microscopic and macroscopic hematuria, assessing serum creatinine levels, assessment of the glomerular filtration rate, histological evaluation of renal biopsy, urinary albumin creatinine ratio, albumin excretion rate, creatinine clearance rate, 24-hour urinary protein secretion, and renal imaging (e.g., MRI, ultrasound).

In some embodiments, the amount of 15-PGDH inhibitor administered to the subject can be an amount effective to induce endogenous renal PGE2 levels of the subject.

In other embodiments, the amount of 15-PGDH inhibitor administered to the subject can be an amount effective to induce renal vasodilatation, enhance resistance to hypoxia, improve renal hemodynamics, decrease renal oxidative stress, reduce renal inflammation, and preserve renal function.

In other embodiments, the amount of 15-PGDH inhibitor administered to the subject is an amount effective to reduce malondialdehyde (MDA) and NGAL levels, attenuate medulla tubular damage, reduce medulla acute tubular necrosis (ATN) and apoptosis, reduces induction of high-mobility group box 1 (HMGB1) and proinflammatory cytokines, induce renal EP4 PGE2 receptors and A2A adenosine receptors in vascular smooth muscle cells that regulate renal arterioles, increase renal cAMP, AMP, and adenosine levels, and/or inhibit induction of creatinine and KIM-1.

In some embodiments, the pharmaceutical composition may be formulated into a parenteral or oral dosage form. The solid dosage form for oral administration may be manufactured by adding excipient, if necessary, together with binder, disintegrants, lubricants, coloring agents, and/or flavoring agents, to the 15-PGDH inhibitors and shaping the resulting mixture into the form of tablets, sugar-coated pills, granules, powder or capsules. The additives that can be added in the composition may be ordinary ones in the art. For example, examples of the excipient include lactose, sucrose, sodium chloride, glucose, starch, calcium carbonate, kaolin, microcrystalline cellulose, silicate and the like. Exemplary binders include water, ethanol, propanol, sweet syrup, sucrose solution, starch solution, gelatin solution, carboxymethylcellulose, hydroxypropyl cellulose, hydroxypropyl starch, methylcellulose, ethylcellulose, shellac, calcium phosphonate and polypyrrolidone. Examples of the disintegrant include dry starch, sodium arginate, agar powder, sodium bicarbonate, calcium carbonate, sodium lauryl sulfate, stearic monoglyceride and lactose. Further, purified talc, stearates, sodium borate, and polyethylene glycol may be used as a lubricant; and sucrose, bitter orange peel, citric acid, tartaric acid, may be used as a flavoring agent. In some embodiments, the pharmaceutical composition can be made into aerosol formulations (e.g., they can be nebulized) to be administered via inhalation.

The 15-PGDH inhibitors described herein may be combined with flavoring agents, buffers, stabilizing agents, and the like and incorporated into oral liquid dosage forms such as solutions, syrups or elixirs in accordance with conventional methods. One example of the buffers may be sodium citrate. Examples of the stabilizing agents include tragacanth, acacia and gelatin.

In some embodiments, the 15-PGDH inhibitors may be incorporated into an injection dosage form, for example, for a subcutaneous, intramuscular or intravenous route by adding thereto pH adjusters, buffers, stabilizing agents, relaxants, topical anesthetics. Examples of the pH adjusters and the buffers include sodium citrate, sodium acetate and sodium phosphate. Examples of the stabilizing agents include sodium pyrosulfite, EDTA, thioglycolic acid and thiolactic acid. The topical anesthetics may be procaine HC_{1,} lidocaine HC₁ and the like. The relaxants may be sodium chloride, glucose and the like.

In other embodiments, the 15-PGDH inhibitors may be incorporated into suppositories in accordance with conventional methods by adding thereto pharmaceutically acceptable carriers that are known in the art, for example, polyethylene glycol, lanolin, cacao butter or fatty acid triglycerides, if necessary, together with surfactants such as Tween.

The pharmaceutical composition may be formulated into various dosage forms as discussed above and then administered through various routes including an oral, inhalational, transdermal, subcutaneous, intravenous or intramuscular route. The dosage can be a pharmaceutically or therapeutically effective amount.

Therapeutically effective dosage amounts of the 15-PGDH inhibitor may be present in varying amounts in various embodiments. For example, in some embodiments, a therapeutically effective amount of the 15-PGDH inhibitor may be an amount ranging from about 10-1000 mg (e.g., about 20 mg-1,000 mg, 30 mg-1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg-600 mg). In some embodiments, the 15-PGDH inhibitor is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, the 15-PGDH inhibitor is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg.

In other embodiments, a therapeutically effective dosage amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight.

In still other embodiments, a therapeutically effective dosage amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g., increased or decreased) over time, depending on the needs of the individual.

In some embodiments, a therapeutically effective dosage may be a dosage of 10 μg/kg/day, 50 μg/kg/day, 100 μg/kg/day, 250 μg/kg/day, 500 μg/kg/day, 1000 μg/kg/day or more. In various embodiments, the amount of the 15-PGDH inhibitor or pharmaceutical salt thereof is sufficient to provide a dosage to a patient of between 0.01 μg/kg and 10 μg/kg; 0.1 μg/kg and 5 μg/kg; 0.1 μg/kg and 1000 μg/kg; 0.1 μg/kg and 900 μg/kg; 0.1 μg/kg and 900 μg/kg; 0.1 μg/kg and 800 μg/kg; 0.1 μg/kg and 700 μg/kg; 0.1 μg/kg and 600 μg/kg; 0.1 μg/kg and 500 μg/kg; or 0.1 μg/kg and 400 μg/kg.

Various embodiments may include differing dosing regimen. In some embodiments, the 15-PGDH inhibitor can be administered via continuous infusion. In some embodiments, the continuous infusion is intravenous. In other embodiments, the continuous infusion is subcutaneous. The dosing regimen for a single subject need not be at a fixed interval, but can be varied over time, depending on the needs of the subject.

In one aspect, a pharmaceutical composition comprising an effective amount of the 15-PGDH inhibitor is administered at least twice. In another aspect, a pharmaceutical composition is administered at least five times. In yet another aspect, a pharmaceutical composition is administered at least 10 times. One of ordinary skill in the art can determine how often to administer the composition based on the particular disease or disorder being treated or how the subject has responded to prior treatments. One of ordinary skill in the art can also determine when to administer a treatment relative to the time that an ischemic reperfusion injury event occurs, including before, after, or both.

In one embodiment, the subject is treated with the 15-PGDH inhibitor prior to the ischemic reperfusion injury event. In one aspect, the subject can be treated starting at least several days before the event or as close to several minutes before the ischemic reperfusion injury event. For example, the 15-PGDH inhibitor therapy can begin at about 2 hours, 8 hours, 24 hours, or 26 hours prior to ischemic reperfusion injury. One of ordinary skill in the art will appreciate that the 15-PGDH inhibitor can be administered at varying times and not just at about 2, 8, 24, or 26 hours prior to ischemic reperfusion injury. In one aspect, the range of time for treating prior to the ischemic reperfusion injury event can be from about 1.0 minutes to about 72 hours. In another aspect, the range of time for treating prior to the ischemic reperfusion injury event can be from about 10 minutes to about 48 hours. In another aspect, the range of time for treating prior to the ischemic reperfusion injury event can be from about 30 minutes to about 24 hours.

In one embodiment, the subject is treated with the 15-PGDH inhibitor after the IRI event or both before and after as described above. In one aspect, the subject can be treated starting immediately after such as several minutes after the ischemic reperfusion ischemic reperfusion injury event. For example, the 15-PGDH inhibitor therapy can begin at about 30 minutes, 2 hours, 8 hours, 24 hours, or 48 hours after the ischemic reperfusion injury. One of ordinary skill in the art will appreciate that the 15-PGDH inhibitor can be administered at varying times as well.

Because the methods of the invention are useful for treating ischemic reperfusion injury, the methods further include treating other diseases and disorders associated with ischemic reperfusion injury, including, but not limited to, myocardial ischemic reperfusion injury and brain ischemic reperfusion injury.

### EXAMPLE

In this Example, we increased endogenous PGE2 levels using a 15-PGDH inhibitor (SW033291), which inhibits the enzymatic function of 15-hydroxyprostaglandin dehydrogenase (15-PGDH) that is responsible for catalyzing the rate-limiting step in PGE2 catabolism. Inhibition of 15-PGDH has been shown to increase endogenous PGE2 and cAMP levels and potentiate tissue repair in several mouse models of injury and disease. The present study showed that increasing endogenous PGE2 level by 15-PGDH inhibition led to renal vasodilation and conferred renal protection against ischemic AM.

Materials and methods Animals

Male C_{57/}BL6 mice (age, 10 weeks; body weight, 20-25 g) were purchased from Orient Bio Inc. (Daejeon, Korea). Before the experiments, all mice were housed individually in standard cages and were allowed to acclimate under specific pathogen-free conditions in the animal care facility of the College of Medicine of Inje University. The care of and experimental procedures involving animals were approved by the Institutional Animal Care and Use Committee of Inje University (Protocol No. 2016-010).

### Renal Ischemia-Reperfusion Injury Model

Mice were anesthetized with isoflurane using a vaporizer and placed on a heating pad to maintain their body temperature at 37° C. Both renal arteries were identified through dorsal incisions and clamped for 20, 30, 35, or 37 minutes. Reperfusion was confirmed visually upon release of the clamps. Surgical wounds were closed, and mice were administered 1 mL of saline i.p. The mice were kept in a warm incubator until they regained consciousness and were allowed to recover with ad libitum access to food and water. SW033291 (18040; Cayman), indomethacin, eglandin (219; Mitsubishi chemical Holdings), PGE2 (P0409; Sigma-Aldrich) each, 5 mg/kg or vehicle was administered three times at 1 hour before, immediately after, and 12 hours after AKI. Serum and kidney tissue were collected 24 hours after renal IRI.

### Measurement of PGE2 Levels

After reperfusion for 24 hours, kidney tissues were harvested, rinsed in ice-cold PBS containing indomethacin (10 μg/mL), and snap-frozen in liquid nitrogen. Next, the kidney tissues (˜20 mg) were homogenized in 500 pL of cold PBS containing indomethacin (10 μg/mL) using a tissue homogenizer. The suspension was sonicated in an ice-water bath for 1 minute using cycles of 10 seconds of sonication with 10 seconds of cooling, and they were then centrifuged for 10 minutes at 12,000 rpm. The supernatant was collected for PGE2 assay. Protein concentrations were determined by BCA assay (23225; Thermo Scientific). The PGE2 level in the supernatant was measured using a PGE2 ELISA Kit (SKGE004B; R&D Systems) in triplicate. PGE2 levels were expressed as ng PGE2/mg protein.

### Assessment of Renal Function

Renal function was assessed by determining the serum levels of creatinine (KB02-H1; Arbor Assays), Lipocalin-2 (NGAL; MLCN20; R&D Systems), and kidney injury molecule-1 (KIM-1; MKM100; R&D Systems) after reperfusion for 24 h.

### Necrotic and Apoptotic Cell Death Assays

To evaluate necrosis, 5-μm-thick paraffin sections were stained with H&E. Tubular injury was scored semi quantitatively according to a scoring system by a pathologist who examined at least 20 separate fields (×400) in the outer medulla, which is the zone most sensitive to ischemic injury. The scoring system was as follows: 0, no damage; 1, patchy isolated unicellular necrosis; 2, tubular necrosis<25%; 3, tubular necrosis 25-50%; and 4, tubular necrosis>50%. At least 20 consecutive high-power fields (magnification, ×400) per section were scored by two operators blind to the details of the experiment. To analyze the frequency of apoptosis, 5-μm-thick paraffin sections were subjected to terminal deoxynucleotidyl transferase-mediated dUTP nick-end labeling (TUNEL) assay (APT110; Millipore) according to the manufacturer's protocol. TUNEL-positive cells were counted in at least five separate fields (×640 magnification) in the outer medulla, and the apoptosis index (%, number of apoptosis cells/total number of cells) was calculated using GENASIS software.

Assessment of Renal Vasodilatation in the Outer Medulla

To quantify vasodilation, the inner arteriole area of the outer medulla was determined using α-SMA-stained sections. After counterstaining with Mayer's hematoxylin, the inner area of α-SMA-positive vessels in the outer medulla (×25) was measured using ImageJ. The results are expressed as average areas of renal arteries outer medulla. The antibodies listed in Table 2.

TABLE 2
A list of antibodies

| Antibody | Sources | Company | Cat. No | dilution |
|---|---|---|---|---|
| Alpha-SMA | Mouse | DAKO | M0851 | 1:500 |
| | monoclonal | | | |
| HMGB1 | Rabbit | abcam | AB18256 | 1:500 |
| | polyclonal | | | |
| β-actin | Mouse | Santa cruz | SC-47778 | 1:1000 |
| | monoclonal | biotechnology | | |
| HRP-conjugated | Goat | BETYL | A120-101P | 1:30000 |
| Rabbit IgG | polyclonal | | | |
| HRP-conjugated | Goat | BETYL | A90-116P | 1:30000 |
| Mouse IgG | polyclonal | | | |

### Measurement of Proinflammatory Cytokine Levels

Inflammatory cytokine mRNA and protein levels were measured by real-time PCR and ELISA, respectively. Kidney tissue and serum were harvested after reperfusion for 24 h. Total RNA was extracted from frozen kidney tissue using TRIzol reagent (15596018; Invitrogen), according to the manufacturer's protocol. RNA was converted to cDNA using oligo-dT primers. IL-17, TNF-α, and IL-1β mRNA levels were determined by real-time PCR with SYBR green PCR Master Mix and the primers listed in Table 1. For ELISA, frozen kidney tissues were homogenized in phosphate buffer. Serum IL-17 (M1700; R&D Systems), TNF-α (MTA00B; R&D Systems), and IL-1β (MLBOOC; R&D Systems) were measured using commercial ELISA kits according to the manufacturer's instructions.

TABLE 1
A list of primers for RT-PCR

| | | | Size | Accession |
|---|---|---|---|---|
| Genes | Forward | Reverse | (bp) | no. |
| TNFα | CCACATC | AGGGTCT | 259 | NM_013693.3 |
| | TCCCTCC | GGGCCAT | | |
| | AGAAAA | AGAACT | | |
| | (SEQ ID | (SEQ ID | | |
| | NO: 1) | NO: 2) | | |
| IL-1β | TCACAGC | TGTCCTC | 284 | NM_008361.4 |
| | AGCACAT | ATCCTGG | | |
| | CAACAA | AAGGTC | | |
| | (SEQ ID | (SEQ ID | | |
| | NO: 3) | NO: 4) | | |
| IL-17a | TCCAGAA | AGCATCT | 239 | NM_010552.3 |
| | GGCCCTC | TCTCGAC | | |
| | AGACTA | CCTGAA | | |
| | (SEQ ID | (SEQ ID | | |
| | NO: 5) | NO: 6) | | |
| IL-4 | CCTCACA | ATCGAAA | 155 | NM_021283.2 |
| | GCAACGA | AGCCCGA | | |
| | AGAACA | AAGAGT | | |
| | (SEQ ID | (SEQ ID | | |
| | NO: 7) | NO: 8) | | |
| IL-10 | CCAAGCC | TTTTCAC | 162 | NM_010548.2 |
| | TTATCGG | AGGGGAG | | |
| | AAATGA | AAATCG | | |
| | (SEQ ID | (SEQ ID | | |
| | NO: 9) | NO: 10) | | |
| IL-24 | CACTCTG | GCTTTCA | 157 | NM_053095.2 |
| | GCCAACA | CCAAAGC | | |
| | ACTTCA | GACTTC | | |
| | (SEQ ID | (SEQ ID | | |
| | NO: 11) | NO: 12) | | |
| GAPDH | TTCACCA | GGCATGG | 237 | NM_008084.3 |
| | CCATGGA | ACTGTGG | | |
| | GAAGGC | TCATGA | | |
| | (SEQ ID | (SEQ ID | | |
| | NO: 13) | NO: 14) | | |

### Measurement of Reactive Oxygen Species Levels

Reactive oxygen species (ROS) levels were determined by measurement of malondialdehyde (MDA), the end product of lipid peroxidation in brain lysates. Free MDA reacts with thiobarbituric acid (TBA) at 95° C. to generate an MDA-TBA adduct, which can be quantified colorimetrically at a wavelength of 532 nm. MDA levels were measured in kidney lysates using a lipid peroxidation (MDA) assay kit (ab118970; Abcam). Results were corrected for total protein level and are expressed as μM MDA/g protein.

### Measurement of cAMP and Adenosine levels

After reperfusion for 24 hours, kidney tissues were harvested, homogenized in 10 volumes of 0.1 M HCl, and centrifuged for 10 minutes at 12,000 rpm. The protein concentration was determined by BCA assay. cAMP levels in kidney tissues were measured using a cAMP Complete ELISA Kit (ADI-900-163; Enzo Life Science) an adenosine Assay Kit (KA4547; abnova) and high-performance liquid chromatography (HPLC).

### Assessment of PGE2 and Adenosine Receptors

PGE2 receptors (EP1, EP2, EP3 and EP4) and adenosine receptor (A_{2A}) mRNA levels were determined by real-time PCR with SYBR green PCR Master Mix and the primers listed in Table 1. Their protein levels were determined by western blotting assay and immunofluorescence analysis. The antibodies listed in Table 2.

### Statistical Analysis

Statistical analyses were performed with one-way analysis of variance (ANOVA) followed by the Bonferroni post-test when three or more experimental groups were compared. Values of p<0.05 were considered indicative of statistical significance. The Kaplan-Meier product-limit method was used to generate survival curves. Survival data were analyzed by the Mantel-Cox log-rank test.

### Results

### 15-PGDH inhibition attenuates renal dysfunction in mice with ischemic AKI

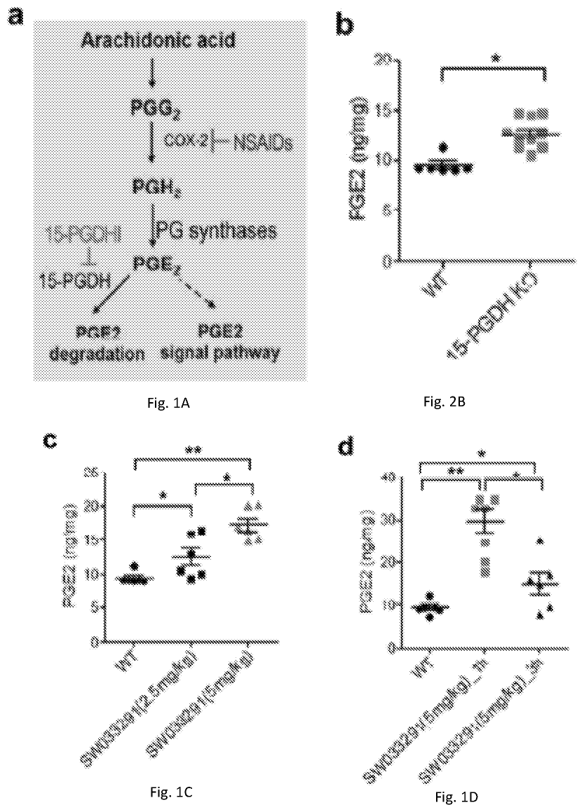

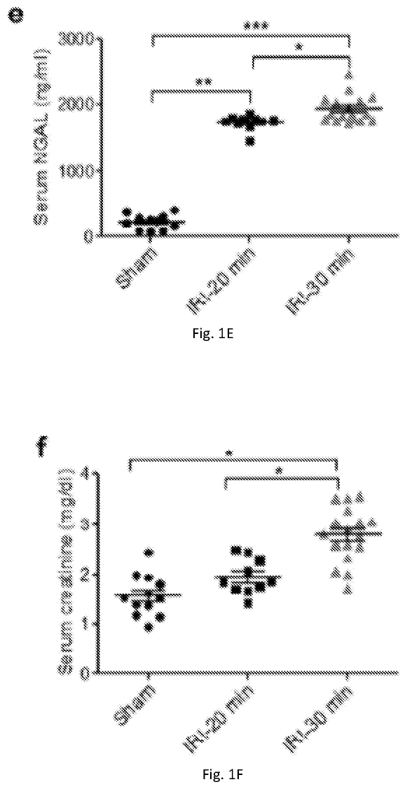

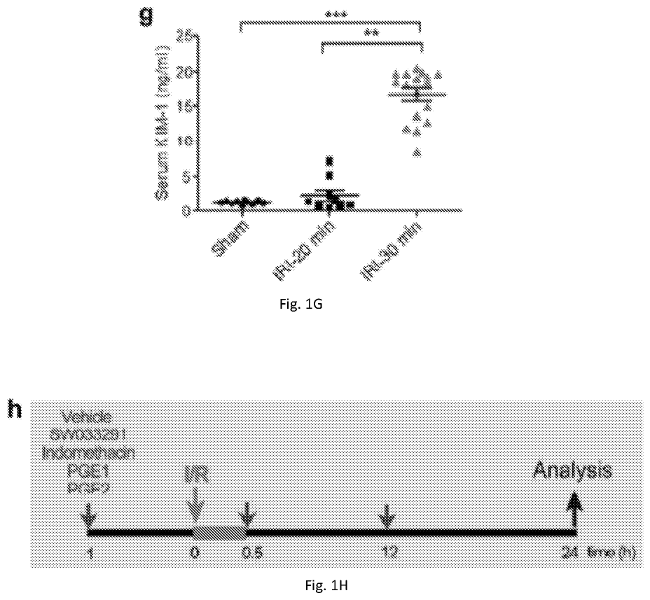

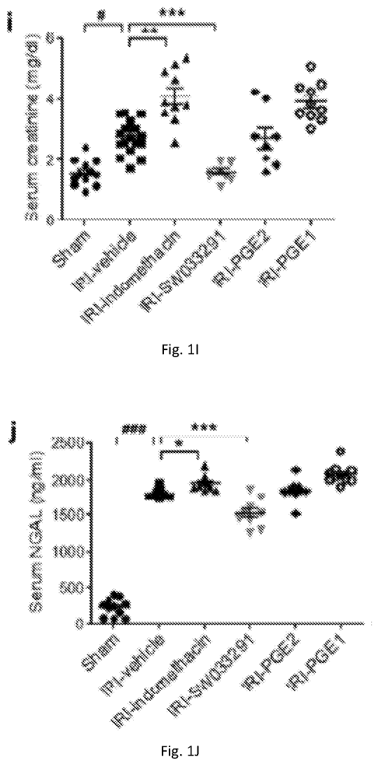

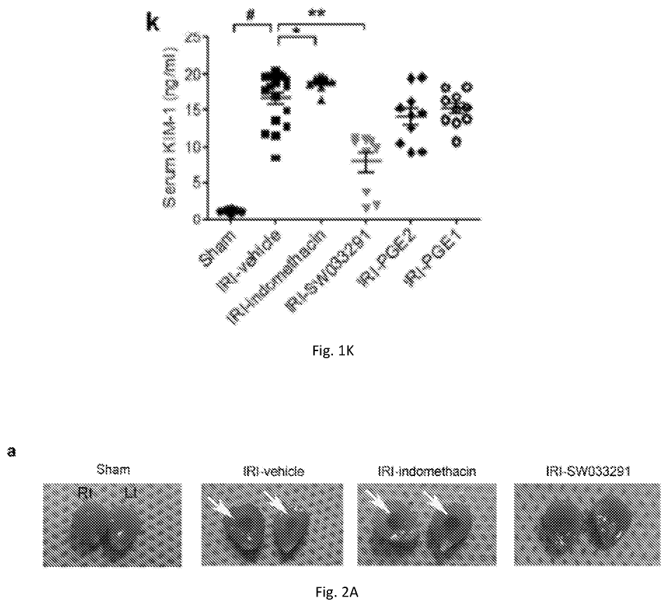

Endogenous PGE2 is synthesized from arachidonic acid by cyclooxygenase (COX) and various synthases and is degraded by 15-hydroxyprostaglandin dehydrogenase (15-PGDH). Endogenous PGE2 levels are reduced by NSAIDs (including those selective for inhibition of COX-2) and are increased by a 15-PGDH inhibitor (SW033291), which inhibits endogenous PGE2 degradation (FIG. 1A). To confirm that 15-PGDH regulates endogenous PGE2 expression in the kidney, we evaluated endogenous PGE2 levels in 15-PGDH knockout (KO) and wild-type (WT) mice. 15-PGDH KO mice exhibited significantly increased endogenous PGE2 levels in kidney tissue (FIG. 1B). Pharmacologic inhibition of 15-PGDH with SW033291 similarly, and in dose-dependent fashion, upregulated endogenous PGE2 levels in kidney tissue at 3 hours after administration of 2.5 or 5 mg/kg SW033291 (FIG. 1C). The level of PGE2 induced by SW033291 (5 mg/kg) peaked at 1 hour at nearly twice as high as at baseline and as at 3 hours post drug injection (FIG. 1D). Mice undergoing 30 min of bilateral ischemic injury (IRI-30 min; moderate injury) exhibited significantly greater ischemic AM compared with control mice, as indicated by increased NGAL, creatinine, and KIM-1 levels, but IRI-20 min (mild injury) did not (FIGS. 1E-G). To determine effects of inhibiting 15-PGDH on protection from renal IRI, mice were subjected to IRI-30 min and were administered 3 doses of vehicle (IRI-vehicle) or SW033291 (IRI-SW033291), 1 hour before, immediately after, and 12 hours after renal IRI (FIG. 1H). As additional comparators, parallel cohorts of mice were administered either, indomethacin, exogenous PGE1, or PGE2 (FIG. 1H). Serum NGAL, creatinine, and KIM-1 levels were determined as markers of renal injury. As expected, IRI-vehicle exhibited significant ischemic AM, as indicated by increases in creatinine, NGAL, and MM-1 (FIGS. 1I-K). However, IRI-SW033291 markedly protected kidney from IRI, significantly reducing creatinine, NGAL and MM-1 as compared to IRI-vehicle animals (FIG. 1I-K). Generating PGE2 in situ within the kidney with SW033291 was more effective than systemic administration of either exogenous PGE1 or PGE2 (FIGS. 1I-K). In contrast, inhibiting endogenous PGE2 production with 3 doses of indomethacin significantly aggravated IRI. In summary, these results suggest that increasing endogenous PGE2 level via a 15-PGDH inhibitor ameliorates renal dysfunction in ischemic AM, whereas renal dysfunction is worsened by inhibition of COX. Administering exogenous PGE1 or PGE2, at typically tolerated doses, is less effective for renal protection than generating PGE2 directly in the kidney through administering SW033291.

### 15-PGDH inhibition ameliorates renal necrosis and apoptosis in mice with ischemic AM

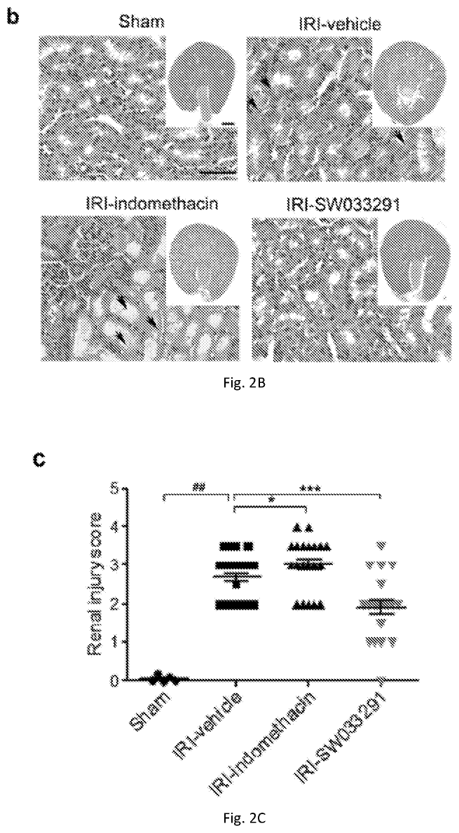

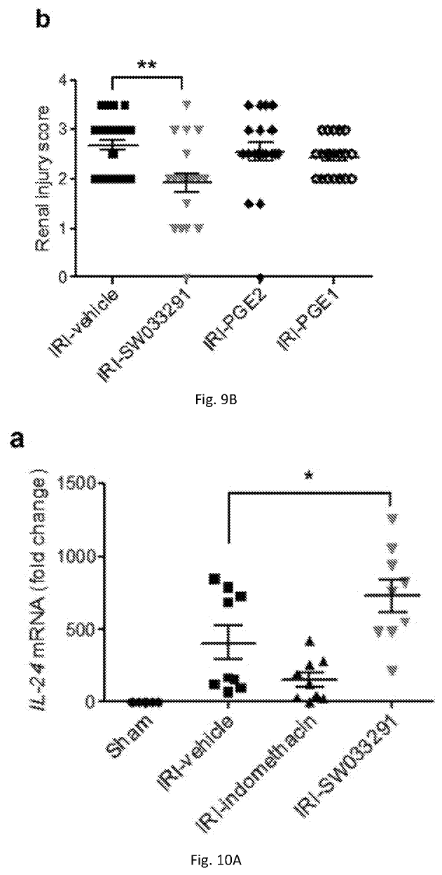

During renal IRI, tubular epithelial cells undergo injury, apoptosis, and acute tubular necrosis (ATN; i.e., AKI resulting in damage to the tubules). As a consequence, post-ischemic congestion persists in the outer medulla and exacerbates renal injury by worsening hypoxia. By gross pathology, IRI-vehicle group mice showed increased tissue congestion in the outer medulla versus sham group mice, which was ameliorated by treating with SW033291 and worsened by treating with indomethacin (FIG. 2A). Histopathology assessment of IRI-vehicle mice revealed features of acute tubular damage with tubular dilatation, extensive tubular necrosis, and apoptosis (FIGS. 2B and D). However, SW033291 treatment markedly alleviated renal injury in the IRI mice, reducing the histologic renal injury score and the count of TUNEL positive apoptotic cells (FIGS. 2C and E). In contrast, IRI-indomethacin group mice showed further exacerbated renal injury. Moreover, generating PGE2 in situ with SW033291 was again more effective than systemic administration of either exogenous PGE1 or PGE2 (FIG. 9). These data suggest that treating mice with 3 doses of 15-PGDH inhibitor, initiated just prior to renal ischemia, attenuates tubular damage in the outer medulla, reducing both ATN and apoptosis.

### 15-PGDH inhibitor treatment suppresses the inflammatory response after ischemic AM

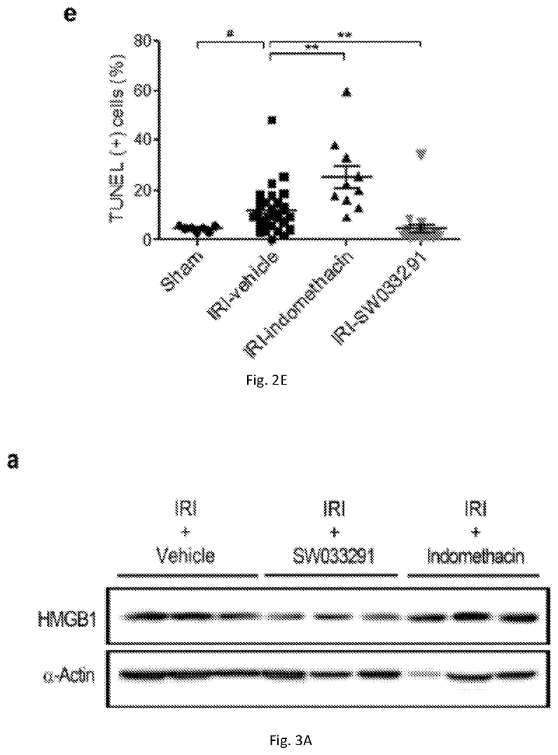

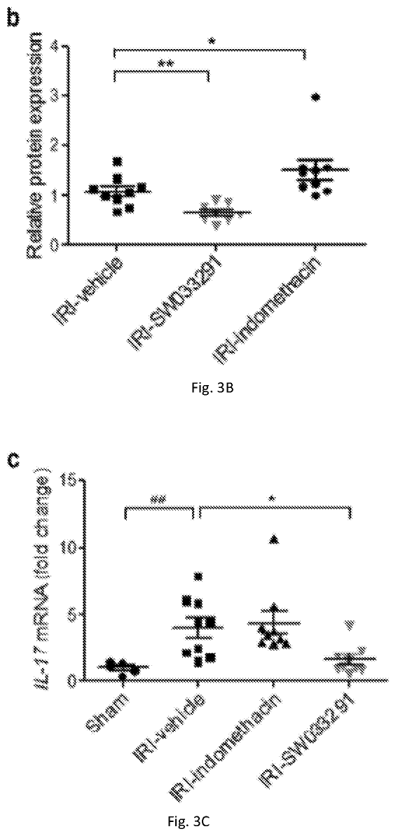

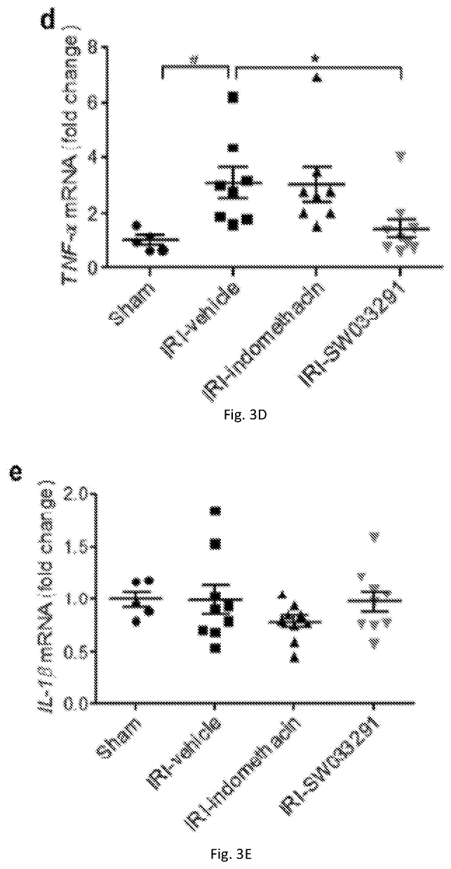

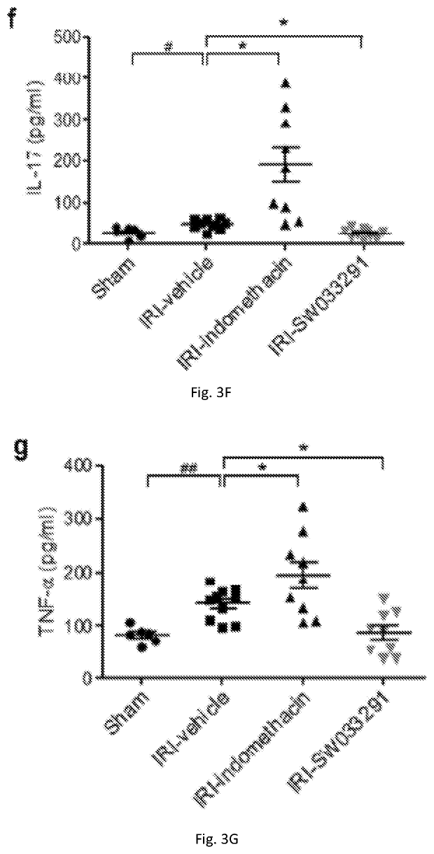

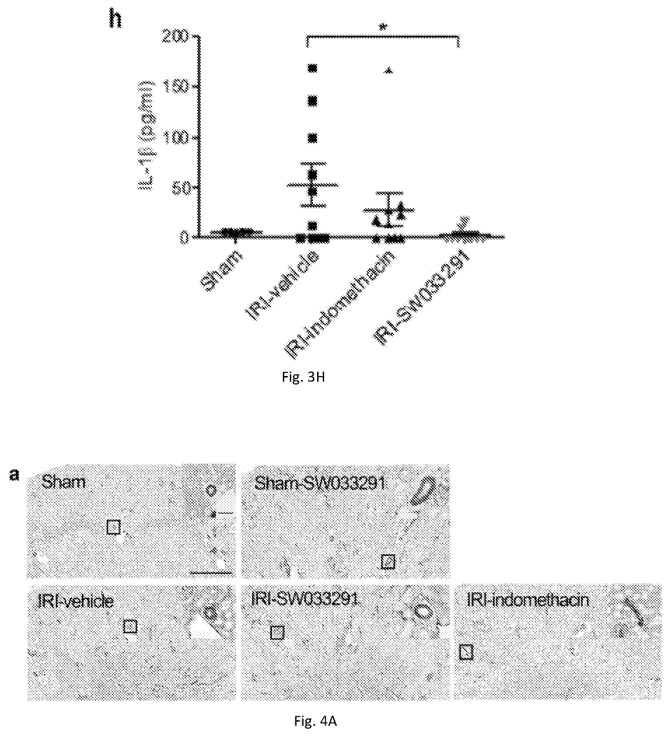

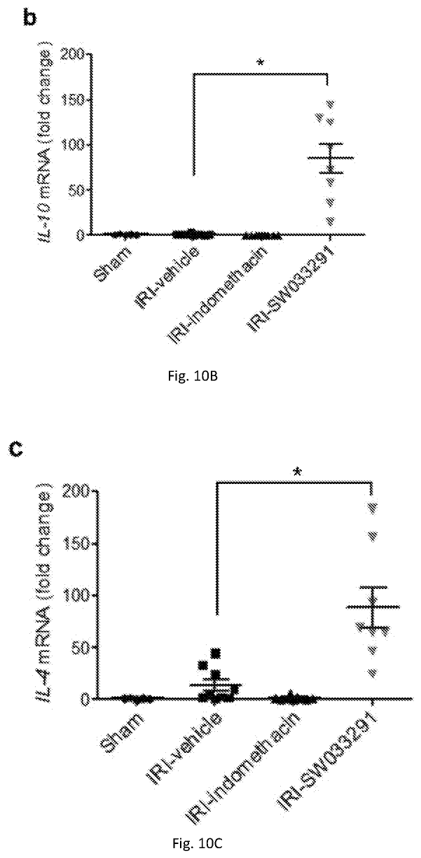

The inflammatory response contributes to the pathology of ischemic AM during the extension phase. High-mobility group box 1 (HMGB1) is a type of danger-associated molecular patterns (DAMPs) molecule and is a key mediator of IRI and an inducer of proinflammatory cytokines. SW033291 treatment reduced the level of HMGB1 protein 30% compared to the IRI-vehicle group, whereas indomethacin treatment increased HMGB1 by 50% (FIGS. 3A and B). To further investigate renal inflammation, we determined the mRNA and protein levels of pro-inflammatory cytokines IL-17, TNF-α, and IL-1β in kidney tissue by real-time PCR and ELISA (FIGS. 3C-H). IRI-vehicle group mice showed significant increases in IL-17 and TNF-α versus the sham group. IL-1β protein also increased, though this did not reach statistical significance (P=0.08). However, the IRI-SW033291 group mice showed blockade of induction of IL-17 and TNF-α; and reductions in IL-1β protein. In contrast, IRI-indomethacin group mice showed increased induction of inflammatory cytokines. SW033291 treatment of IRI mice additionally significantly induces the anti-inflammatory cytokine IL-4, IL-10 and its related family member IL-24 (FIG. 10). These data suggest that treatment with a 15-PGDH inhibitor reduces induction of both HMGB1 and proinflammatory cytokines in parallel with preventing damage to renal cells.

### 15-PGDH Inhibitor Treatment Induces Renal Vasodilatation in the Outer Medulla Pari Passu with Induction of a cAMP/AMP/Adenosine Signaling Pathway

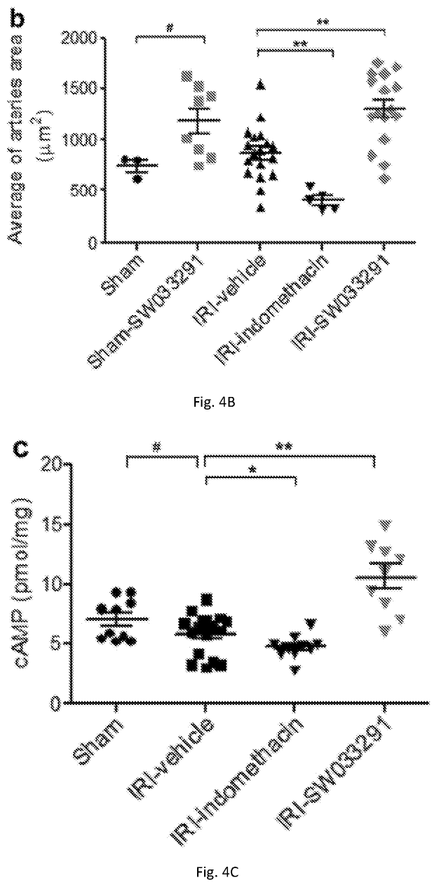

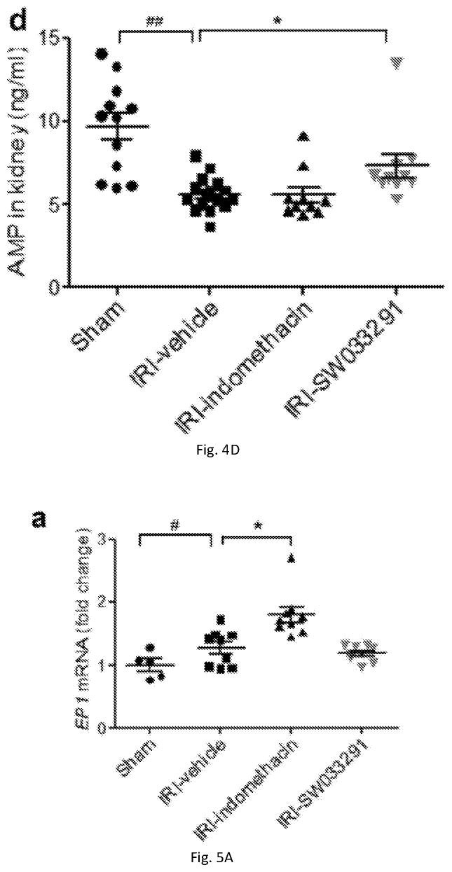

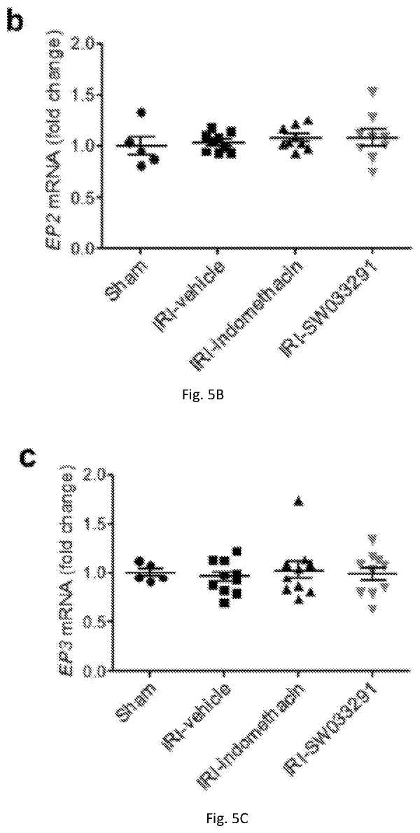

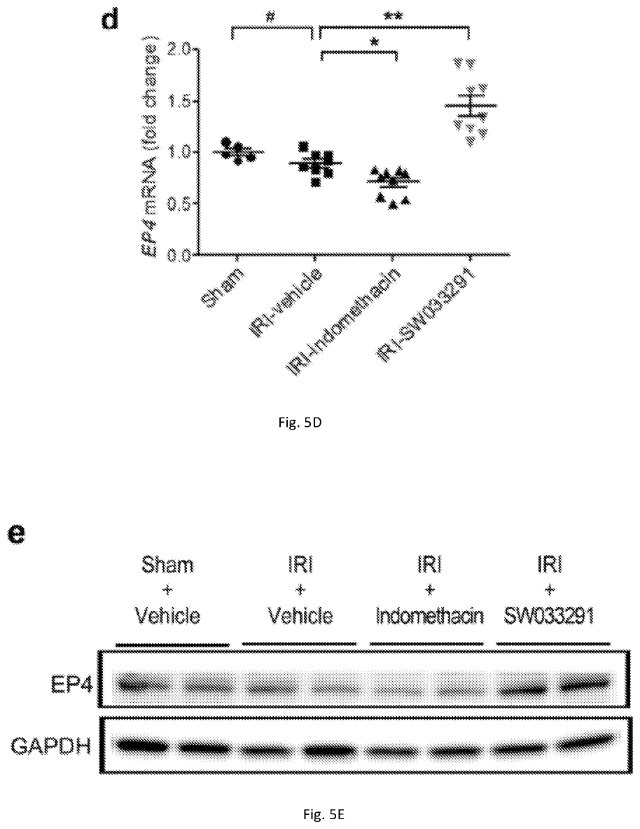

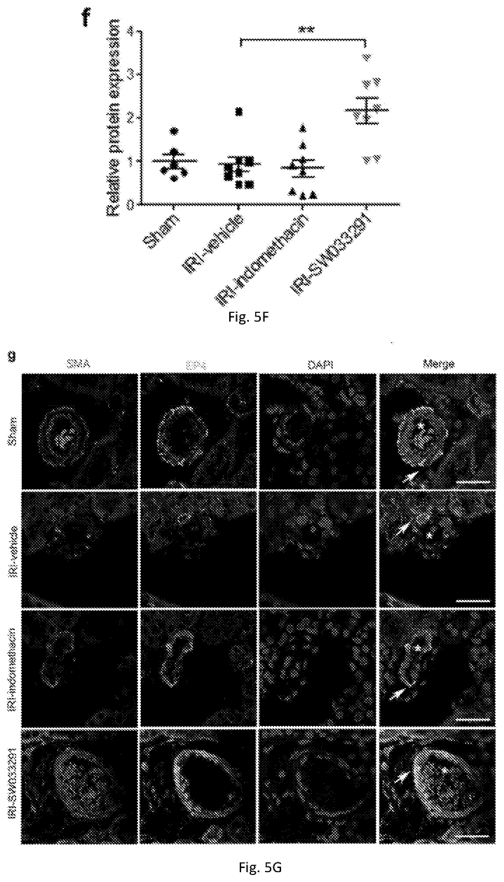

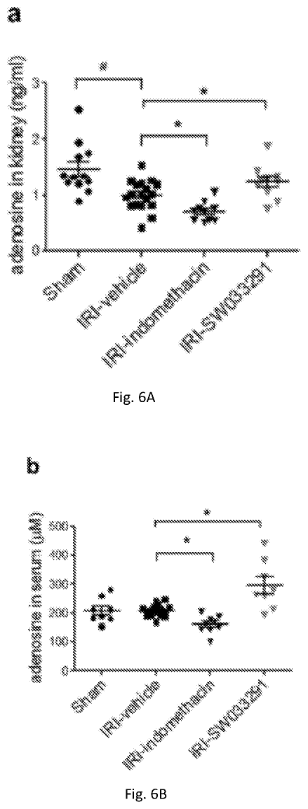

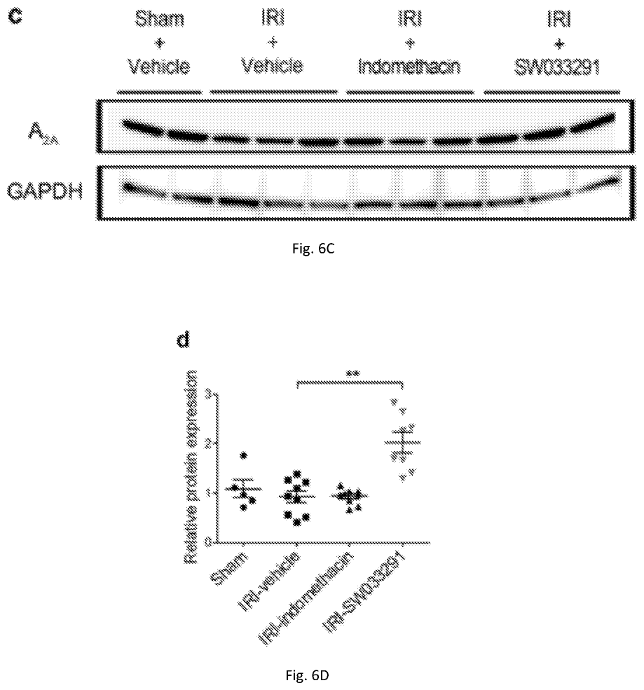

As 15-PGDH inhibitor treatment attenuated multiple measures of ischemic renal injury, we directly assessed its effects on morphology of the renal microvasculature as determined by quantitating the a-smooth muscle actin (α-SMA)-positive renal arteriole area in the outer medulla. The average of renal arteriole area in the IRI-vehicle group was similar to that in the sham mice. However, renal arteriole area of IRI mice was significantly increased by treatment with SW033291; whereas, it was significantly decreased by treatment with indomethacin (FIGS. 4A and B). Interestingly, the average of renal arteriole area of normal mice was also increased by administration of SW033291 (FIGS. 4A and B). It is known that the vasodilation effect of endogenous PGE2 is mediated by a cAMP-dependent mechanism in the renal afferent arteriole. In addition, adenosine is a recognized mediator of renal vasodilation. Levels of cAMP and AMP, derivatives of adenosine, were all significantly decreased in IRI-vehicle group mice compared to the sham group, but these changes were substantially reversed by treating IRI mice with SW033291 (FIGS. 4C and D). Similarly, levels of adenosine in the kidney were reduced by 29% in IRI mice, but also were increased by SW033291 (FIG. 6A). SW033291 moreover significantly increased levels of serum adenosine (FIG. 6B). To further interrogate SW033291 effects on these signaling pathways, we additionally characterized the drug's effects on the PGE2 and adenosine receptors. In comparison to sham and to IRI mice, SW033291 significantly increased EP4 receptor mRNA and protein levels (by up to 2.3-fold) (FIGS. 5D-F), without effecting EP1, 2 or 3. Indomethacin, in contrast, reduced EP4 mRNA, but increased by 40%, levels of mRNA for EP1, a receptor known to be involved vasoconstriction. Moreover, in comparison to sham and to IRI mice, SW0332391 also induced levels of the adenosine A_{2A} receptor protein (FIGS. 6C and D) Immunohistochemistry showed SW033291 induction of both EP4 and A_{2A} receptors was localized to alpha-SMA positive vascular smooth muscle cells (VSMCs) that directly regulate constriction or dilation of renal arterioles (FIG. 5G; FIG. 6E). Thus induction of renal vasodilation by 15-PGDH inhibition is well correlated with induction of downstream mediators that include EP4, cAMP (that is a known product of PGE2 stimulation of EP4), adenosine, and A_{2A} adenosine receptors, with induction of both the EP4 PGE2 receptors and the adenosine A_{2A} receptors targeted to VSMCs.

Pretreatment with a single 15-PGDH Inhibitor dose mitigates renal dysfunction

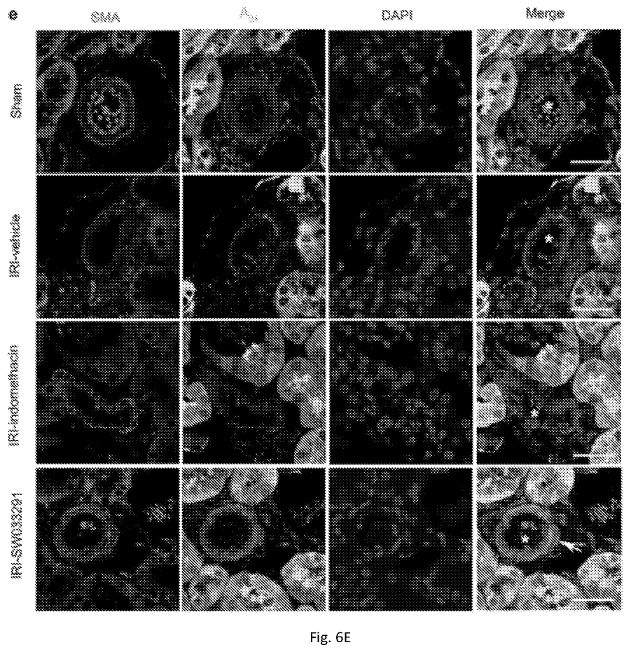

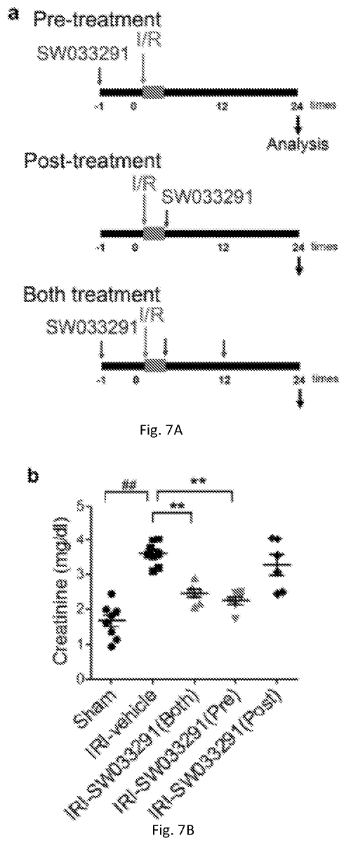

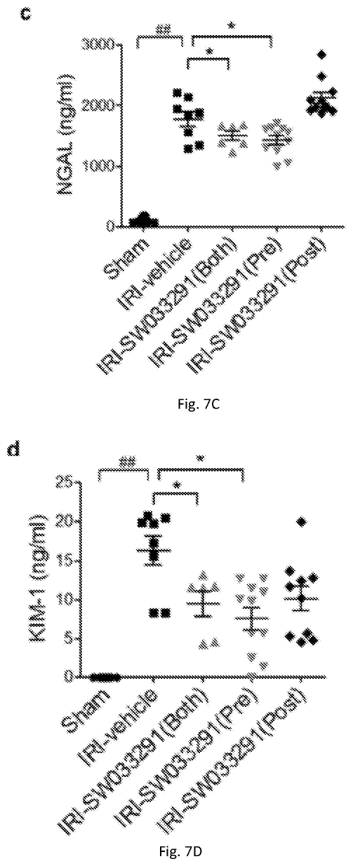

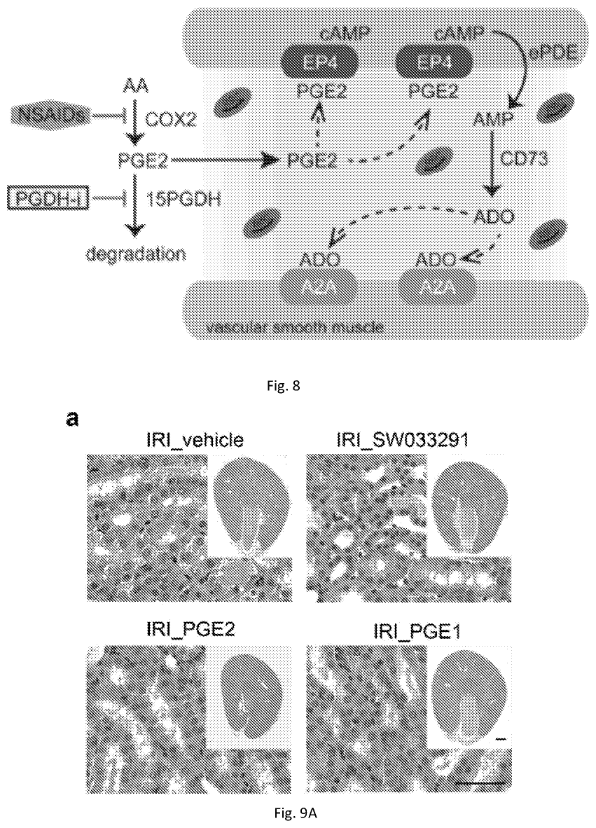

To gain further insight into the contributions of the individual doses in the 3 dose schedule of administering SW033291, we compared the effects of administering just a single dose of SW033291 administered 24 hours before IRI (Pre); versus a single dose administered immediately after IRI (Post); versus our standard 3 doses given 24 hours before, immediately after, and 12 hours after IRI (Both) (FIG. 7A). Surprisingly, a single pre-IRI dose of SW033291 was as effective in ameliorating AKI as the full 3 dose regimen (FIG. 7B-D). These findings suggest that a single dose of SW033291 administered prior to IRI can provide prophylaxis from inducing AKI. Short term post IRI treatment with just one dose SW033291 was, in the absence of concomitant pretreatment drug, insufficient to ameliorate AM (FIGS. 7B-D).

Pretreatment with a single 15-PGDH Inhibitor dose attenuates AKI induced oxidative stress and blocks injury induced increases in renal PGE2

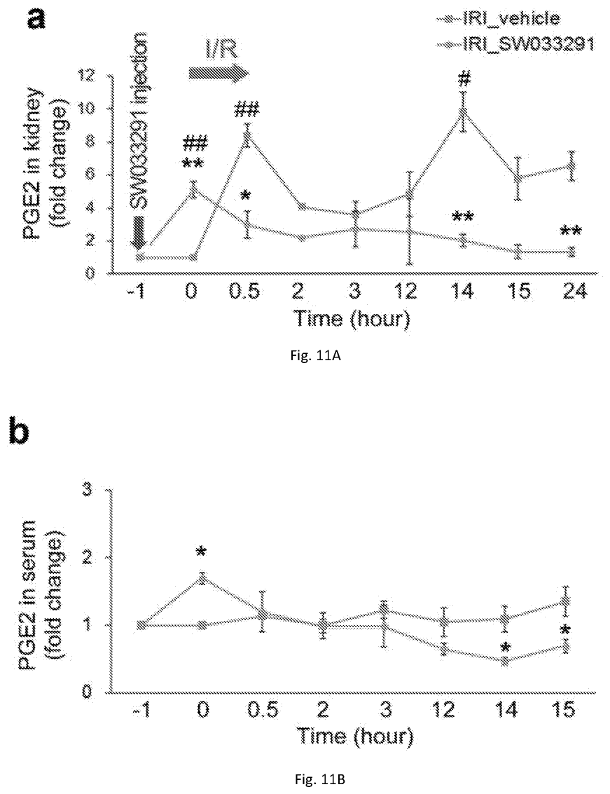

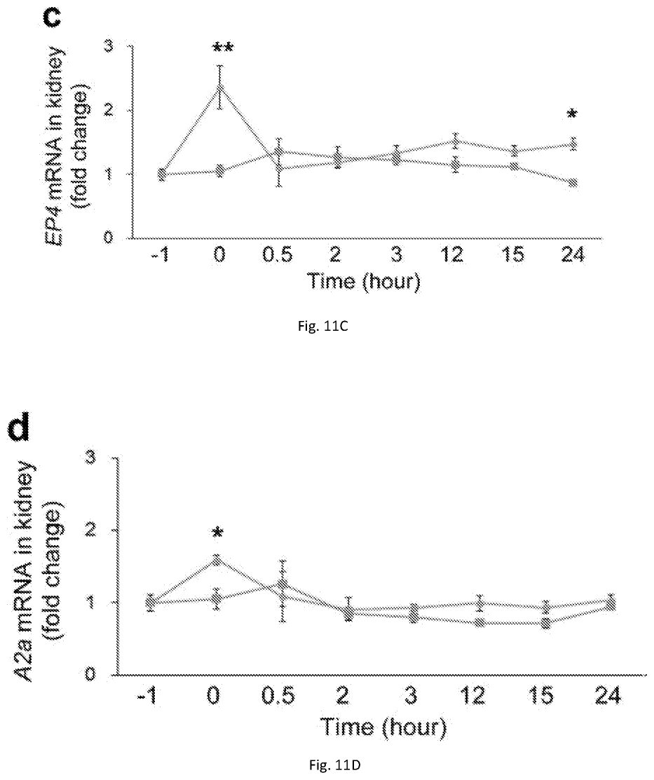

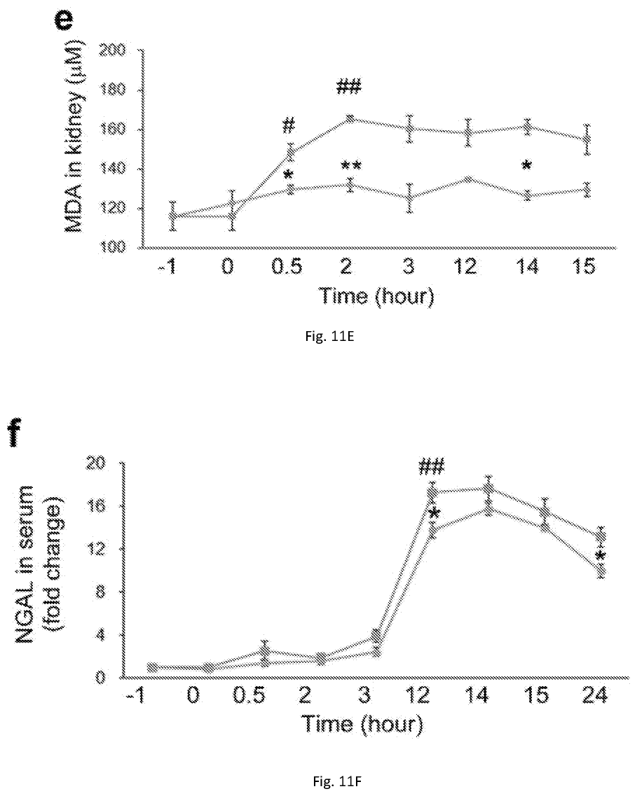

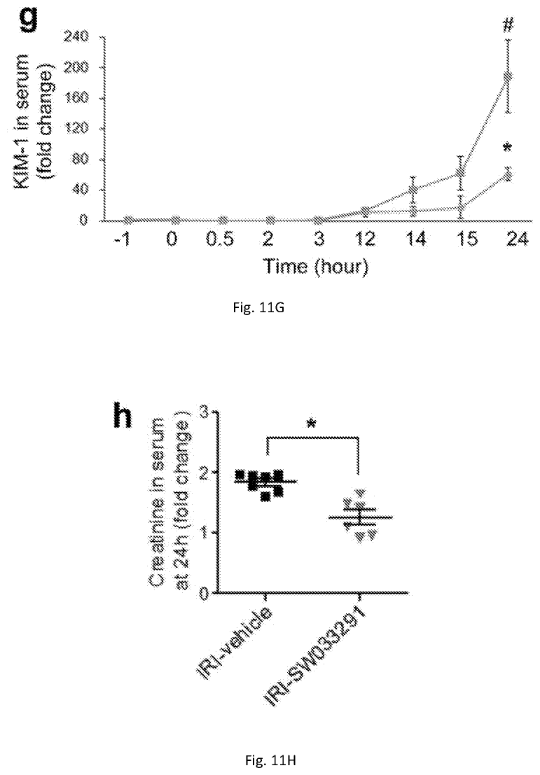

To gain further insight into the mechanism of prophylaxis of ischemic AM by single-dose 15-PGDH inhibitor pre-treatment, we examined the drug's effect on the time course of induction of various markers of AKI. In IRI mice, malondialdehyde (MDA), a marker of oxidative stress, began to increase immediately after renal IRI and peaked at 2 hours at 48% above baseline; however, a single pre-IRI dose of SW033291 blunted and reduced this increase to only 14% (FIG. 11E). NGAL began to increase at 3 hours in IRI mice and peaked at 12 hours at 17.28-fold above baseline, and pretreatment with SW033291 reduced this peak induction by 20% (FIG. 3F). Moreover, IRI-SW033291 group mice showed notably effective blockade of induction of MM-1 and of creatinine (FIG. 3G and H). Intriguingly, renal PGE2 also showed induction by renal injury, demonstrating two peaks in the IRI mice, an immediate post-IRI peak of 8.41-fold over baseline and a 14 hour post-IRI peak of 9.83-fold over baseline (FIG. 11A). A single pre-IRI dose of SW033291 induced a PGE2 peak of 5.12-fold over baseline before IRI, and was sufficient to substantially block the two post-IRI peaks of PGE2 at 0.5 and 14 hours (FIG. 11A). Moreover, SW033291 prophylaxis significantly decreased serum PGE2 level of BI30 mice at 24 hours (FIG. 11B). Furthermore, pretreatment with SW033291 promoted the increase of EP4 and A2A receptors by 2.4-fold and 1.6-fold over baseline at 0.5 hours (FIGS. 11C and D). Thus, prophylactic induction of endogenous PGE2 with 15-PGDH inhibitor before IRI induces vasodilatation, induces PGE2 EP4 receptors and adenosine A2A receptors, significantly attenuated post IRI oxidative stress, and thereby reduced multiple markers of renal injury. Moreover, a 5-fold prophylactic induction of endogenous PGE2 blocked the substantially greater 8- to 10-fold post IRI injury induced “catch-up” induction of the PGE2 injury repair signal.

### 15-PGDH Inhibitor Treatment is Non-Toxic and Promotes Recovery after Renal IRI

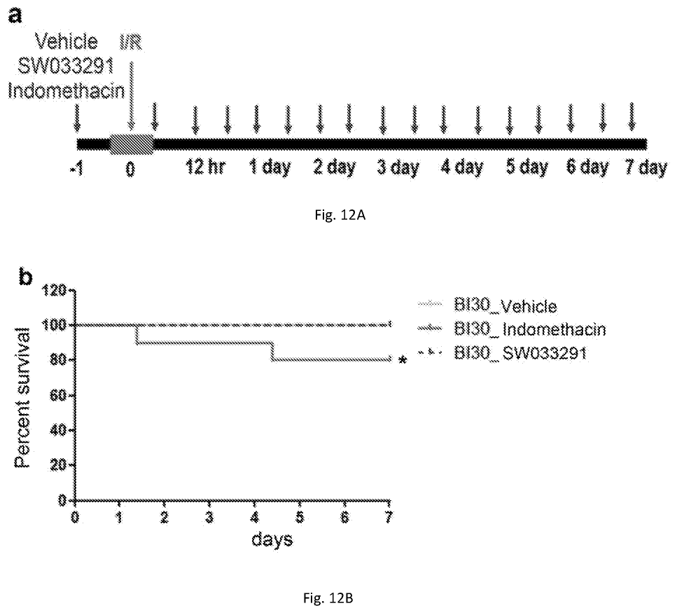

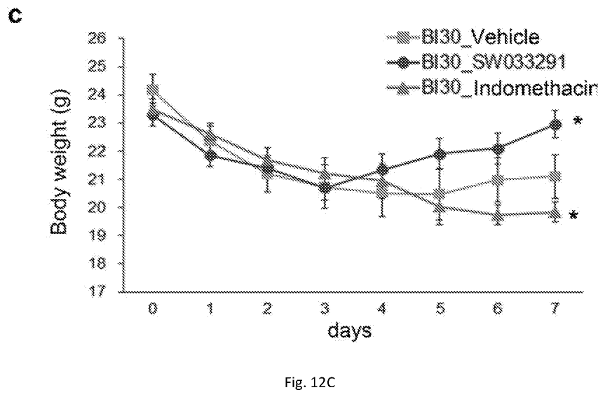

To assess for any potential toxicity of SW033291 in the setting of AKI, we examined survival and daily body weight over 7 days following ischemic AKI induced by bilaterally clamping the renal arteries for 30 minutes, followed by reperfusion, in mice in which the duration of SW033291 treatment was extended by administering the drug once pre-IRI followed by twice daily for 7 days after AKI (FIG. 12A). All BI30-vehicle group and B130-SW033291 group mice survived at 7 days. However, SW033291 treated mice regained greater weights than did vehicle treated mice at POD7 (FIGS. 12B and C). Thus, no evidence for toxicity was noted on prolonged administration of SW033291. Supporting that the beneficial effects of SW033291 are due to modulation of renal PGE2, BI30-indomethacin group showed 25% reduced survival and 6% lower body weight on POD7 than did mice injected with vehicle control (FIGS. 12B and C).

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A method for preventing or treating renal ischemia reperfusion injury or acute kidney injury associated with renal ischemia reperfusion injury in a subject in need thereof, the method comprising:
administering to the subject a therapeutically effective amount of a 15-PGDH inhibitor.

2. The method of claim 1, wherein the method prevents or treats acute kidney injury associated with renal ischemia reperfusion injury.

3. The method of claim 1, wherein the amount of 15-PGDH inhibitor administered to the subject is an amount effective to induce endogenous renal PGE2 levels of the subject.

4. The method of claim 1, wherein the amount of 15-PGDH inhibitor administered to the subject is an amount effective to induce renal vasodilatation, enhance resistance to hypoxia, improve renal hemodynamics, decrease renal oxidative stress, reduce renal inflammation, and/or preserve renal function.

5. The method of claim 1, the amount of 15-PGDH inhibitor administered to the subject is an amount effective to reduce malondialdehyde (MDA) and NGAL levels, attenuate medulla tubular damage, reduce medulla acute tubular necrosis (ATN) and apoptosis, reduces induction of high-mobility group box 1 (HMGB1) and proinflammatory cytokines, induce renal EP4 PGE2 receptors and A2A adenosine receptors in vascular smooth muscle cells that regulate renal arterioles, increase renal cAMP, AMP, and adenosine levels, and/or inhibit induction of creatinine and KIM-1.

6. The method of claim 1, wherein the 15-PGDH inhibitor is administered before the ischemia reperfusion injury.

7. The method of claim 6, wherein the 15-PGDH inhibitor is administered at a range of about 1 minute to about 72 hours before the ischemia reperfusion injury.

8. The method of claim 6, wherein the 15-PGDH inhibitor is administered at a range of about 10 minutes to about 48 hours before the ischemia reperfusion injury.

9. The method of claim 6, wherein the 15-PGDH inhibitor is administered at a range of about 30 minutes to about 36 hours before the ischemia reperfusion injury.

10. The method of claim 6, wherein the 15-PGDH inhibitor is administered at a time selected from the group consisting of 2 hours, 8 hours, 24 hours, and 26 hours before the ischemia reperfusion injury.

11. The method of claim 1, wherein said ischemia reperfusion injury is associated with a transplant in said subject.

12. The method of claim 11, wherein said transplant is a kidney transplant.

13. The method of claim 1, wherein said ischemia reperfusion injury is associated with cardiovascular surgery or sepsis.

14. The method of any of claims 1 to 13, wherein the 15-PGDH inhibitor has the following formula (V): 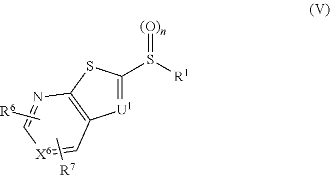
wherein n is 0-2
X⁶ is independently is N or CR^{c}
R¹, R⁶, R⁷, and R^{c} are each independently selected from the group consisting of hydrogen, substituted or unsubstituted C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, alkyl amino substituted with hydroxyl, C₅-C₂₀ aryl amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, phosphate esters, groups incorporating amino acids or other moieties expected to bear positive or negative charge at physiological pH, combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂-CH₂X, CH₂—CH₂—CH₂X, O—CH₂—CH₂X, X, (wherein X=H, F, Cl, Br, or I), CN, (C═O)—R′, (C═O)N(R′)₂, (CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, Cl, Br, or I), CN, (C═O)—R′, (C═O)N(R′)₂, COOR′ (wherein R′ is H or a lower alkyl group), and R³ or R⁴ may be absent;
or a pharmaceutically acceptable salt, tautomer, or solvate thereof.

15. The method of any of claims 1 to 13, wherein the 15-PGDH inhibitor has the following formula following formula: 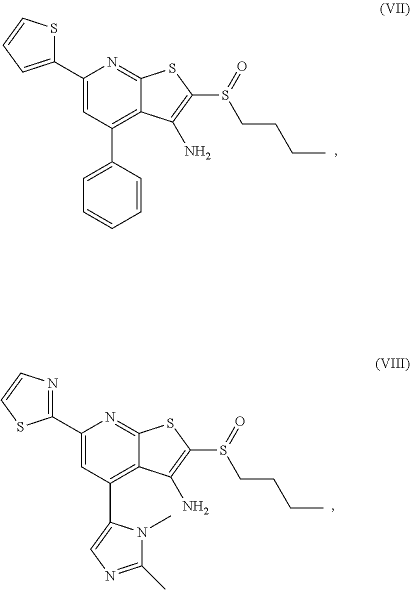 [SMILES: CCCCS(=O)c1sc2nc(-c3cccs3)cc(-c3ccccc3)c2c1N ; CCCCS(=O)c1sc2nc(-c3nccs3)cc(-c3cnc(C)n3C)c2c1N]
or a pharmaceutically acceptable salt, tautomer, or solvate thereof.
